(19) United States
(12) Patent Application Publication
(10) Pub. No.: US 2003/0055511 A1
(43) Pub. Date: Mar. 20, 2003

(54) Shaped particle comprised of bone material and method of making the particle

(76) Inventors: Jeffrey E. Schryver, Cordova, TN (US); Michael B. Cooper, Memphis, TN (US); Keith M. Kinnane, Bartlett, TN (US); Marc Long, Memphis, TN (US); Trevor Allen, York (GB); Ed Margerrison, York (GB); Robert Morgan, (US); Julie A. Bearcroft, Bartlett, TN (US); Andrew Harrison, York (GB); William B. Kaiser, Sunnyvale, CA (US)

Correspondence Address: Smith & Nephew, Inc., 1450 Brooks Road, Memphis, TN, 38116, US

(21) Appl. No.: 10/099,616

(22) Filed: Mar. 15, 2002

Related U.S. Application Data
(63) Continuation-in-part of application No. 09/517,981, filed on Mar. 3, 2000.
(63) Continuation-in-part of application No. 09/792,681, filed on Feb. 23, 2001.

(51) Int. Cl. A61F 2/28

(52) U.S. Cl. 623/23.5; 623/23.63; 623/16.11; 623/919

(57) ABSTRACT

A shaped particle for use in an array of interlocking particles to repair, replace, improve or augment a bone deficiency is provided. The particle is comprised of bone material and, in a preferred embodiment, has six extremities, and the interstitial spaces between the extremities of one particle accept the extremities of an adjacent particle in an array. In a preferred embodiment, the bone material is demineralized bone material. In some embodiments, the particle is suspended in a material that facilitates application of the particle to bone, and the material may contain biological factors that augment bone growth or prevent infection.

### CROSS-REFERENCE TO RELATED APPLICATIONS

The present application is a Continuation-in-Part Application of U.S. patent application Ser. No. 09/517,981 filed Mar. 3, 2000 and a Continuation-in-Part Application of U.S. patent application Ser. No. 09/792,681 filed Feb. 23, 2001, both of which are incorporated by reference herein in their entirety.

### FIELD OF THE INVENTION

The present invention generally relates to a shaped particle, and its manufacture, as a bone graft substitute (BGS) and the use of such a substitute to repair, replace, augment or improve a bone deficiency, wherein the particle is preferably comprised of a bone material, such as a demineralized bone material. The invention also relates to a composition having such a particle in a suspension material to enhance the utility of the particle as a bone graft substitute.

### BACKGROUND OF THE INVENTION

Bone graft is used to fill spaces in bone tissue that are the result of trauma, disease degeneration or other loss of tissue. Clinicians perform bone graft procedures for a variety of reasons, often to fill a bone void created by a loss of bone or compaction of cancellous bone. In many instances the clinician also must rely on the bone graft material to provide some mechanical support, as in the case of subchondral bone replacement or compaction grafting around total joint replacement devices. In these instances, clinicians pack the material into the defect to create a stable platform to support the surrounding tissue and hardware.

There are several options available to the orthopedic clinician for bone graft material. Most commonly, the source of the graft material is either the patient (autograft) or a donor (allograft). In autograft and, to a lesser extent, in allograft there are biological factors such as proteins or cells that are present that can assist in the fracture healing process. Xenografts and bone graft substitutes are other options.

Autograft is taken from the patient's own body and is the most commonly used graft material. The graft, which can come in the form of chips or blocks, is harvested from an ectopic bone site within the body, such as the iliac crest, and used in the deficient site. Autograft has the potential draw back of increased pain and morbidity associated with a second surgical procedure, in addition to having a limited supply of the bone.

Allograft is another form of graft which comes from human bone tissue donated to tissue banks, such as from a cadaver. Allograft is available in a number of forms: granules or chips, blocks or struts, and processed forms such as gels or putties. In addition to having a limited supply, a serious drawback of allograft is the risk of disease transmission.

Xenografts are one such choice which come from non-human bone-tissue donors and are often processed and mixed with other components such as hydroxyapatite or other calcium salts. Again, xenografts are not favored for human use because of concerns over disease transmission and immunogenicity.

Given the disadvantages associated with autograft and allograft, many have focused efforts on developing new synthetic bone substitute materials that can fill the existing need.

The biological and physical demands placed on a bone graft material vary in response to the treatment indication. For instance, clinicians prefer different physical forms of the materials (granules, blocks, dense, porous, putty/paste, cement) depending on the difficulty filling a bone void sufficiently with graft. Craniomaxillofacial defects typically pose relatively low load-bearing requirements on the graft material. The size of the defect may influence whether a conductive graft is sufficient or if an inductive graft is required. In some instances, a graft's ability to withstand high load and maintain structural support over a long period of time (such as in the case of compaction grafting around a revision joint prosthesis) is more important than the graft's ability to accelerate bone healing or bridge a gap (such as in the case of grafting to achieve spine fusion). For this reason, it is important to have more adaptable materials for bone graft over products currently available in the art, which fall short of easily conforming to a multitude of applications. Use of such a product would have the inherent advantage of being less costly and more efficient for personnel in orthopedics.

Two properties associated with currently available synthetic granules have inherent disadvantages. First, it is difficult to get the granules from the package into the defect. The granules are generally small, less than 10 mm in any one dimension, and difficult to grasp individually. The granules have no means to form an aggregate, so clinicians cannot handle them in unison. Secondly, if the granules spill into an open surgical wound, the granules stick to soft tissue, which makes it difficult to clear them from the wound. Clinicians fear that if left in the wound, the granules can cause further complications such as migration into the articulating surfaces, potentially causing further damage.

Synthetic bone graft granules are commonly supplied in a simple glass vial, and very little has been done to improve the handling characteristics or ease the surgical procedure. There are a few exceptions. Although a syringe-like device is available on the market to assist in delivery of granules to the graft site, this does not address the issue of preferential sticking of the granules to soft tissue in the wound. Alternatively, demineralizing allograft products are commercially available which come premixed in a gel or putty for improved handling.

The BGS materials that have been used commercially exhibit various levels of bioactivity and various rates of dissolution. BGS products are currently available in several forms: powder, gel, slurry/putty, tablet, chips, morsels, and pellet, in addition to shaped products (sticks, sheets, and blocks). In many instances, the form of BGS products is dictated by the material from which they are made. Synthetic materials (such as calcium sulfates or calcium phosphates) have been processed into several shapes (tablets, beads, pellets, sticks, sheets, and blocks) and may contain additives such as antibiotics (e.g., Osteoset®-T (Wright Medical Technology; Arlington, Tenn.)) or bioactive agents. Allograft products, in which the source of the bone graft material is a donor, are typically available as chips and can be mixed with a gel to form a composite gel or putty. None of the current products and technologies offered for BGS is capable of offering an allograft granule or shape for easy delivery and scaffold structure, in addition to being conformable to the surgical site. Furthermore, none but two (Osteoset®-T and OP-1) of the current products and technologies offered for BGS is capable of offering an allograft or synthetic granule or shape containing a bioactive agent or agents, such as an antibiotic or bone morphogenetic proteins.

Past solutions to produce BGS products have included gel, putty, paste, formable strips, blocks, granules, chips, pellets, tablets, and powder. A skilled artisan recognizes there are multiple references directed to bone graft substitutes, including Medica Data International, Inc., Report #RP-591149, Chapter 3: Applications for Bone Replacement Biomaterials and Biological Bone Growth Factors (2000) and Orthopaedic Network News, Vol. 11, No 4, October 2000, pp. 8-10.

To date, DBM products have been produced in chips, granules, powder, gel, or putty forms only. No solid DBM product (as opposed to a putty) which has undergone a shaping process is currently available to the health care provider. It is a disadvantage of the presently available products to have no shape which is interlocking, and the irregularly-shaped chips of presently available products do not compact sufficiently and also fail to generate reproducible results. Other calcium sulfate-based products have been made using a casting or molding process, as opposed to a dry powder compaction process of the present invention. Osteoset®-T pellets are likely to have been tableted because of their simple shape. A more complicated shape that could provide improved interlocking between the granules over the tableting process used in the art requires the use of a more advanced manufacturing process. In some embodiments, the manufacturing of JAX® (Smith+Nephew, Inc.; Memphis, Tenn.) bone void filler requires the use of a powder compaction process to be able to produce the advanced interlocking granule shape.

The following table compares the embodiments of the present invention to those in the related art.

| | | | Comparison to |
|---|---|---|---|
| | | Manufacturer/ | Present |
| Category | Product | Distributor | Invention |
| Allograft | Tricortical Strips | American Red Cross | Not processed |
| | | Sulzer Spine-Tech | into shape |
| | | Allosource | |
| | | National Tissue Bank | |
| | Cancellous chips | American Red Cross | Not processed |
| | | Allosource | into shape |
| | | National Tissue Bank | |
| | Cortical/cancellous | American Red Cross | Not processed |
| | chips | Sulzer Spine-Tech | into shape |
| | | Allosource | |
| | | National Tissue Bank | |
| | Small Implants | Dowels MD-Series | Machined from |
| | | (RTI) | bone |
| DBM | Gel | Grafton (Osteotech) | Not processed |
| | | Dynagraft (Gensci | into shape |
| | | Regeneration | |
| | | Laboratories) | |
| | Putty | Grafton (Osteotech) | Not processed |
| | | OrthoBlast, Dynagraft | into shape |
| | | (Gensci Regeneration | |
| | | Laboratories) | |
| | | Allomatrix (Wright | |
| | | Medical Technology) | |
| | | DBX (Synthes) | |
| | Paste | OrthoBlast (Gensci | Not processed |
| | | Regeneration | into shape |
| | | Laboratories) | |
| | | Osteofil (Medtronic | |
| | | Sofamor Danek) | |
| | | DBX (Synthes) | |
| | | Regenafil (RTI) | |
| | “Crunch” | Grafton (Osteotech) | Not processed |
| | | | into shape |
| | Formable strip | OpteForm (Exactech) | Thermoplastic/ |
| | | Regenaform (RTI) | thermo- |
| | | | formable |
| | | | polymer carrier |
| Bone | Blocks, | Pro-Osteon 200, 500 | Harvested from |
| Substitutes | granules/chips | (Interpore) | marine coral |
| | (coralline | | |
| | hydroxyapatite) | | |
| | Granules/chips | Pro-Osteon 500R | Harvested from |
| | (calcium carbonate | (Interpore) | marine coral; |
| | w/calcium | | patented |
| | phosphate outer | | process |
| | layer) | | (sintering) |
| | Formable strip | Collagraft (Zimmer) | Sintered HA |
| | (bovine collagen | | mixed |
| | with | | w/collagen on |
| | hydroxyapatite and | | site |
| | tricalcium | | |
| | phosphate) | | |
| | Strip (collagen and | Healos (Orquest - non | Sintered HA |
| | hydroxyapatite | US/Sulzer Spine-Tech) | coated |
| | matrix) | Healos/MP52 | w/collagen; |
| | | (Orquest - non US/ | Impregnated |
| | | Sulzer Spine-Tech) | w/BMP |
| | Pellets/tablets | Osteoset (Wright | Tableted, but |
| | (calcium sulfate | Medical Technology) | may be molded |
| | hemihydrate) | | (proprietary |
| | | | information) |
| | Pellets/tablets | Osteoset T (Wright | Tableted, but |
| | (calcium sulfate | Medical Technology) | may be molded |
| | hemihydrate with | | (proprietary |
| | Tobramycin | | information) |
| | Sulfate) | | |
| | Pellets/tablets | Stimulan | May be molded |
| | (calcium sulfate) | (Biocomposites/ | and/or tableted |
| | | Encore Orthopedics) | (proprietary |
| | | Profusion (Bio- | information) |
| | | Generation, Inc.) | |
| | | Possibly (Howmedica | |
| | | Osteonics Corp.) | |
| | Paste (calcium | Alpha-BSM (ETEX - | May be |
| | phosphate) | non US) Norian SRS | sintered |
| | | | (proprietary |
| | | | information) |
| | Paste | CORTOSS | Polymer matrix |
| | (bioglass/ceramic) | (Orthovita - | |
| | | non US) | |
| | Porous Blocks | VITOSS (Orthovita) | May be |
| | (calcium | | sintered |
| | phosphate) | | (proprietary |
| | | | information) |
| | Small Implants | RHAKOSS | Unknown - but |
| | | (Orthovita - not | not compacted |
| | | commercial) | (proprietary |
| | | | information) |
| | Gel (fibroblast | Ossigel (Orquest) | Not processed |
| | growth factor and | | into shape |
| | hyaluronic acid) | | |
| | Powder (calcium | BonePlast (Interpore) | Not processed |
| | sulfate) mixed | BVF Kit (Wright | into shape |
| | with saline | Medical Technology) | |
| | intraoperatively | | |

Other bone graft substitutes are known in the art. U.S. Pat. No. 5,676,700 is directed to interlocking structural elements for augmentation or replacement of bone in which at least four posts of the element project from a hub such that no more than two of the directions of any of the posts lie in a common plane. The elements have posts with oval cross-sections and in a preferred embodiment have an angle of 109.47 degrees between each post.

U.S. Pat. No. 5,178,201 is directed to an implant method, as opposed to a graft method, in which particles with from four to eight pins which extend radially from a center have at least three pins which adhere to a basic pattern. The body diameter of the particle is a maximum of 3 mm, and the specification does not teach tapering of the pins.

U.S. Pat. No. 5,458,970 teaches shaped particles comprising deformed fibers in which the fiber is a zinc oxide whisker having a plurality of needle-like portions being maximally 0.1 mm in length and extending from its nucleus portion.

U.S. Pat. No. 5,258,028 is directed to an injectable micro-implantation system utilizing textured micro particles maximally 3 mm in diameter and having a number of outwardly projecting pillar members.

WO 94/08912 teaches an aggregate having six arms in which the arms are generally obelisk-shaped and have four sides each.

U.S. Pat. Nos. 6,030,636; 5,807,567; and 5,614,206 are directed to calcium sulfate controlled release matrix. They provide forming a pellet prepared by the process comprising mixing powder consisting essentially of alpha-calcium sulfate hemihydrate, a solution comprising water, and, optionally, an additive and a powder consisting essentially of beta-calcium sulfate hemihydrate to form a mixture, and forming the mixture into the pellet. The pellets were formed by pouring a slurry mixture of the desired components into cylindrical molds.

U.S. Pat. Nos. 5,569,308 and 5,366,507 regard methods for use in bone tissue regeneration utilizing a conventional graft material/barrier material layered scheme. The barrier material is a paste formed immediately prior to its use by mixing calcium sulfate powder with any biocompatible, sterile liquid, whereas the graft material is also a paste form comprised of a mixture of water and at least autogenous cancellous bone, DFDBA, autogenous cortical bone chips, or hydroxylapatite.

U.S. Pat. No. 4,619,655 is directed to Plaster of Paris as a bioresorbable scaffold in implants for bone repair. The inventors provide an animal implant composed of a binder lattice or scaffold of Plaster of Paris and a non-bioresorbable calcium material such as calcium phosphate ceramic particles and, in a specific embodiment, the implant may contain an active medicament bound within the plaster. The implant composition of the invention may be preformed into the desired shape or shapes or it may be made up as a dry mix which can be moistened with water just prior to use to provide a fluid or semisolid, injectable formulation which can be injected into the appropriate body space as required for bone reconstruction.

U.S. Pat. No. 4,384,834 is directed to devices for compacting powder into a solid body, comprising a compaction chamber, a moveable support for the powder which extends into the compaction chamber, and means for launching a punch against the powder to form the solid body. The compaction chamber is formed by a block having a conical bore and a conical sleeve having a continuous uncut sidewall moveable within the conical bore to be radially compressed thereby.

U.S. Pat. No. 5,449,481 concerns apparatus and methods for producing a powder compact comprising loading a rubber mold having a cavity shaped according to a desired configuration of the powder compact into a recess formed by a die, in addition to a lower punch inserted into the die. The method steps include filling a cavity of the rubber mold with powder, placing an upper punch in contact with an opposing surface of the die, and pressing the rubber mold filled with powder in a space formed by the die, the lower punch and the upper punch. In specific embodiments, the upper or lower punches are secured.

U.S. Pat. No. 5,762,978 is directed to a batching device having a series of die holes which are fed powder or granular material, upper and lower punches for each die hole, wherein the punches have counterfacing respective working heads, in addition to a rotary turret comprising the die holes, and driving means for adjusting distances between the working heads of the punches. The driving means includes a driving cam for at least one of the punches and filling operation cam means.

U.S. Pat. No. 6,106,267 regards tooling for a press for making an ingestible compression molded product, such as a tablet, from a granular feedstock material wherein the tooling comprises a die having a cylindrical die cavity and an open end for introducing the feedstock, and first and second punches with end faces which compress the feedstock material and which thereby would form the product whose surfaces conform to the end faces of the punches. The tip portion of the first punch is formed of an elastically deformable material so as to undergo deformation upon compression of the feedstock and which includes a wiping ring for wiping the inner surface of the die cavity upon movement of the punch within the die.

U.S. Pat. No. 5,603,880 concerns methods and an apparatus for manufacturing tablets. Plastic polymer film is pressed to form receptacles and filled with a predetermined amount of a powder under a pressurized condition.

U.S. Pat. No. 6,177,125 regards methods for manufacturing coated tablets from tablet cores and coating granulate using a press having at least one compression chamber and a feed device for tablet cores, comprising adding a pasty tablet core to the coating granulate to be compressed and compressing the coating granulate and the tablet cores simultaneously in a single pressing step.

U.S. Pat. No. 5,654,003 is directed to methods of making a solid comestible by forming deformable particles in size from 150 to 2000 microns wherein the particles are compressible in a die and punch tableting machine by subjecting a feedstock comprising a sugar carrier material, wherein the compressed product possesses a rigid structure and has a hard surface which resists penetration and deformation.

U.S. Pat. No. 5,017,122 regards a rotary tablet press for molding tablets through compression of powders and granules having a plurality of dies which rotate around a central axis, multiple upper and lower punches rotatable with the dies, and means for introducing electrically charged lubricant particles onto the tablets.

U.S. Pat. No. 5,158,728 is directed to an apparatus for forming a two-layer tablet having a die table comprising multiple die stations, each die having a cylindrical cavity. The upper punch and lower punch has at least one insert sized and positioned on the upper punch means and lower punch means, respectively, to fit within the die cavity on the die on die table.

Thus, presently available compositions and methods in the art provide no bone graft substitute particles having consistent shapes and whose shapes interrelate in a manner to impart a three-dimensional structure for strength and bone ingrowth. The present invention supplies a long-sought solution in the art by making BGS products or granules, such as demineralized bone matrix, by powder compaction to provide a scaffold structure for ingrowth from the host bone and for the purpose of easy delivery.

U.S. Pat. No. 5,290,558 regards a flowable demineralized bone powder composition for bone repair. The composition comprises a bone growth-inducing amount of demineralized osteogenic bone powder in a biocompatible carrier such as glycerol.

U.S. Pat. No. 6,294,187 is directed to a load-bearing osteoimplant comprising a shaped compressed composition of bone particles having a bulk density of greater than about 0.7 g/cm³ and a wet compressive strength of at least about 3 MPa.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### SUMMARY OF THE INVENTION

In an embodiment of the present invention, there is a shaped particle for use in treating a bone deficiency wherein said particle is shaped for use in an array of particles interlocked with one another, comprising a center portion; and at least four tapered extremities projecting from said center portion wherein said projections provide for interstitial spaces between adjacent extremities, each extremity having a base attached at said center portion, an opposite point, a length, and a circular transverse cross-sectional configuration, wherein said interstitial spaces of one said particle will accept at least one extremity of an adjacent said particle to facilitate interlocking of adjacent particles in said array, wherein the particle is comprised of bone material. In a specific embodiment, at least three of said extremities lie in a plane. In another specific embodiment, the particle has six extremities.

In an additional embodiment of the present invention, there is a shaped particle for use in treating a bone deficiency wherein said particle is shaped for use in an array of particles interlocked with one another, comprising a center portion, at least two noncurved extremities, and at least three curved extremities projecting from said center portion wherein said projections provide for interstitial spaces between adjacent extremities, each extremity having a base attached at said center portion, an opposite point, a length, and a transverse cross-sectional configuration, wherein said interstitial spaces of one said particle will accept at least one extremity of an adjacent said particle to facilitate interlocking of adjacent particles in said array, wherein the particle is comprised of a bone material.

In another embodiment of the present invention, there is a shaped particle for use in treating a bone deficiency wherein said particle is shaped for use in an array of particles interlocked with one another, comprising a multi-ring structure having at least four curved projections wherein said projections provide for interstitial spaces between adjacent said projections, and wherein said projections facilitate interlocking of adjacent particles in said array, wherein the particle is comprised of a bone material. In a specific embodiment, bone material is allograft bone material. In another specific embodiment, the allograft bone material is cortical-cancellous bone, cortical bone, cancellous bone, demineralized bone material, or mixtures thereof. In a further specific embodiment, the demineralized bone material is fully demineralized, partially demineralized, or a mixture thereof. In another specific embodiment, the demineralized bone material is a powder. In an additional specific embodiment, the particle has maximum dimensions of about 3-10 millimeters. In an additional specific embodiment, the particle has a maximum dimensions of about 4-8 millimeters. In an additional specific embodiment, the particle has a maximum dimensions of about 4-6 millimeters.

In a specific embodiment, the particle further comprises a biological agent, such as a growth factor, an antibiotic, a strontium salt, a fluoride salt, a magnesium salt, a sodium salt, a bone morphogenetic factor, an angiogenic factor, a chemotherapeutic agent, a pain killer, a bisphosphonate, a growth factor binding/accessory protein, a cell, and a bone growth agent. In a specific embodiment, the growth factor is selected from the group consisting of platelet derived growth factor (PDGF), transforming growth factor □ (TGF-□), insulin-related growth factor-I (IGF-I), insulin-related growth factor-II (IGF-II), fibroblast growth factor (FGF), beta-2- microglobulin (BDGF II), nerve growth factor (NGF), epidermal growth factor (EGF), keratinocyte growth factor (KGF), and bone morphogenetic protein (BMP). In a further specific embodiment, the antibiotic is selected from the group consisting of tetracycline hydrochloride, vancomycin, cephalosporins, quinolone, and aminoglycocides. In a specific embodiment, the quinolone is ciprofloxacin. In a specific embodiment, the aminoglycocide is tobramycin or gentamicin.

In an additional specific embodiment, the bone morphogenetic factor is selected from the group consisting of proteins of demineralized bone, demineralized bone matrix (DBM), bone protein (BP), bone morphogenetic protein (BMP), osteonectin, osteocalcin and osteogenin. In another specific embodiment, the angiogenic factor is monobutyrin, erucimide, synthetic thymosin Beta 4(TB4), synthetic peptide analogs to heparin binding proteins, nicotine, nicotinamide, spermine, angiogenic lipids, thrombin, a related analog/peptide of thrombin, dibutyrin, tributyrin, VEGF, butyric acid, or ascorbic acid. In a further specific embodiment, the angiogenic factor is monobutyrin, erucimide, synthetic thymosin Beta 4(TB4), synthetic peptide analogs to heparin binding proteins, nicotine, nicotinamide, spermine, angiogenic lipids, thrombin, a related analog/peptide of thrombin, dibutyrin, tributyrin, VEGF, butyric acid, ascorbic acid, or derivatives thereof. In a specific embodiment, the growth factor binding/accessory protein is selected from the group consisting of follistatin, osteonectin, sog, chordin, dan, cyr61, thrombospondin, type IIa collagen, endoglin, cp12, nell, crim, acid-1 glycoprotein, and alpha-2HS glycoprotein.

In a specific embodiment, the cell is selected from the group consisting of osteoblasts, endothelial cells, fibroblasts, adipocytes, myoblasts, mesenchymal stem cells, chondrocytes, multipotent stem cells, pluripotent stem cells and totipotent stem cells, and musculoskeletal progenitor cells. In another specific embodiment, the chemotherapeutic agent is selected from the group consisting of cis-platinum, ifosfamide, methotrexate and doxorubicin hydrochloride. In a specific embodiment, the pain killer is selected from the group consisting of lidocaine hydrochloride, bipivacaine hydrochloride, and non-steroidal anti-inflammatory drugs. In a further specific embodiment, the pain killer is a non-steroidal anti-inflammatory drug is ketorolac tromethamine.

In an embodiment of the present invention, there is an array containing multiple shaped particles as described herein. In a specific embodiment, the multiple particles are in a mixture of particles comprised of different materials. In a further specific embodiment, the different materials are selected from the group consisting of bone material, ceramic, calcium salt, bioactive glass, polymer, polymer/ceramic composite, polymer/glass composite, and mixtures thereof. In another specific embodiment, the bone material is an allograft material, such as demineralized bone material, cortical-cancellous bone, cortical bone, cancellous bone, or mixtures thereof. In a specific embodiment, the demineralized bone material is fully demineralized, partially demineralized, or mixtures thereof. In a specific embodiment, the treatment of a bone deficiency is selected from the group consisting of augmentation of bone, repair of bone, replacement of bone, improvement of bone, strengthening of bone and healing of bone. In a specific embodiment, the bone deficiency is selected from the group consisting of a fracture, break, loss of bone, weak bone, brittle bone, hole in bone, void in bone, disease of bone and degeneration of bone. In a further specific embodiment, the disease is selected from the group consisting of osteoporosis, Paget's disease, fibrous dysplasia, osteodystrophia, periodontal disease, osteopenia, osteopetrosis, primary hyperparathyroidism, hypophosphatasia, fibrous dysplasia, osteogenesis imperfecta, myeloma bone disease and bone malignancy.

In a specific embodiment of the present invention, the interlocking of the adjacent particles in the array as described herein provides adequate porosity to allow ingrowth from a host bone. In a specific embodiment, the porosity is between about 40% and about 80%. In another specific embodiment, the porosity is between about 50% and about 80%.

In an additional embodiment of the present invention, there is an array of shaped particles wherein said array comprises a plurality of shaped particles, said shaped particles comprising a center portion; and at least four tapered extremities projecting from said center portion wherein said projections provide for interstitial spaces between adjacent extremities, each extremity having a base attached at said center portion, an opposite point, a length, and a circular transverse cross-sectional configuration, wherein said interstitial spaces of one said particle will accept at least one extremity of an adjacent said particle to facilitate interlocking of adjacent particles in said array of shaped particles, wherein said array of shaped particles provides for treating a bone deficiency, wherein at least one of the particles is comprised of bone material.

In an embodiment of the present invention, there is an array of shaped particles wherein the array comprises a plurality of shaped particles comprising one or more shaped particles from the group consisting of a first shaped particle comprising a center portion and at least four tapered extremities projecting from said center portion wherein said projections provide for interstitial spaces between adjacent extremities, each extremity having a base attached at said center portion, an opposite point, a length, and a circular transverse cross-sectional configuration, wherein said interstitial spaces of one said particle will accept at least one extremity of an adjacent said particle to facilitate interlocking of adjacent particles in said array of shaped particles; a second shaped particle comprising a center portion, at least two noncurved extremities, and at least three curved extremities projecting from said center portion wherein said projections provide for interstitial spaces between adjacent extremities, each extremity having a base attached at said center portion, an opposite point, a length, and a transverse cross-sectional configuration, wherein said interstitial spaces of one said particle will accept at least one extremity of an adjacent said particle to facilitate interlocking of adjacent particles in said array; and a third shaped particle comprising a multi-ring structure having at least four curved projections wherein said projections provide for interstitial spaces between adjacent said projections, and wherein said projections facilitate interlocking of adjacent particles in said array, wherein at least one of the particles is comprised of bone material.

In another embodiment of the present invention, there is a shaped particle for use in treating a bone deficiency wherein said particle is shaped for use in an array of particles interlocked with one another, comprising a multi-ring structure having at least four curved projections wherein said projections provide for interstitial spaces between adjacent said projections, and wherein said projections facilitate interlocking of adjacent particles in said array, wherein the particle is comprised of bone material. In a specific embodiment, the angles between said curved projections are equal.

In an additional embodiment of the present invention, there is a composition for use in treating a bone deficiency comprising a suspension material; and a shaped particle selected from the group consisting of a first shaped particle comprising a center portion and at least four tapered extremities projecting from said center portion wherein said projections provide for interstitial spaces between adjacent extremities, each extremity having a base attached at said center portion, an opposite point, a length, and a circular transverse cross-sectional configuration, wherein said interstitial spaces of one said particle will accept at least one extremity of an adjacent said particle to facilitate interlocking of adjacent particles in said array of shaped particles; a second shaped particle comprising a center portion, at least two noncurved extremities, and at least three curved extremities projecting from said center portion wherein said projections provide for interstitial spaces between adjacent extremities, each extremity having a base attached at said center portion, an opposite point, a length, and a transverse cross-sectional configuration, wherein said interstitial spaces of one said particle will accept at least one extremity of an adjacent said particle to facilitate interlocking of adjacent particles in said array; a third shaped particle comprising a multi-ring structure having at least four curved projections wherein said projections provide for interstitial spaces between adjacent said projections, and wherein said projections facilitate interlocking of adjacent particles in said array, and mixtures thereof, wherein the particle is comprised of bone material.

In a specific embodiment, the suspension material is selected from the group consisting of starch, sugar, glycerin, blood, bone marrow, autograft material, allograft material, fibrin clot and fibrin matrix. In another specific embodiment, suspension material is a binder capable of forming a gel. In an additional specific embodiment, the binder is selected from the group consisting of collagen derivative, cellulose derivative, methylcellulose, hydroxypropylcellulose, hydroxypropylmethyl cellulose, carboxymethylcellulose, fibrin clot, fibrin matrix, hyaluronic acid, chitosan gel, and a biological adhesive such as cryoprecipitate. In a further specific embodiment, the material further comprises a biological agent, as described elsewhere herein.

In a specific embodiment, the composition further includes a clotting factor composition, such as fibrinogen, thrombin, Factor XIII, or a combination thereof.

In an additional embodiment of the present invention, there is a method to treat a bone deficiency comprising the step of applying a shaped particle to a bone deficiency wherein said shaped particle is selected from the group consisting of a first shaped particle comprising a center portion and at least four tapered extremities projecting from said center portion wherein said projections provide for interstitial spaces between adjacent extremities, each extremity having a base attached at said center portion, an opposite point, a length, and a circular transverse cross-sectional configuration, wherein said interstitial spaces of one said particle will accept at least one extremity of an adjacent said particle to facilitate interlocking of adjacent particles in said array of shaped particles; a second shaped particle comprising a center portion, at least two noncurved extremities, and at least three curved extremities projecting from said center portion wherein said projections provide for interstitial spaces between adjacent extremities, each extremity having a base attached at said center portion, an opposite point, a length, and a transverse cross-sectional configuration, wherein said interstitial spaces of one said particle will accept at least one extremity of an adjacent said particle to facilitate interlocking of adjacent particles in said array; and a third shaped particle comprising a multi-ring structure having at least four curved projections wherein said projections provide for interstitial spaces between adjacent said projections, and wherein said projections facilitate interlocking of adjacent particles in said array, wherein the particle is comprised of bone material.

In another embodiment of the present invention, there is a method to treat a bone deficiency comprising the steps of combining a shaped particle with a suspension material wherein said particle is comprised of bone material and is selected from the group consisting of a first shaped particle comprising a center portion and at least four tapered extremities projecting from said center portion wherein said projections provide for interstitial spaces between adjacent extremities, each extremity having a base attached at said center portion, an opposite point, a length, and a circular transverse cross-sectional configuration, wherein said interstitial spaces of one said particle will accept at least one extremity of an adjacent said particle to facilitate interlocking of adjacent particles in said array of shaped particles; a second shaped particle comprising a center portion, at least two noncurved extremities, and at least three curved extremities projecting from said center portion wherein said projections provide for interstitial spaces between adjacent extremities, each extremity having a base attached at said center portion, an opposite point, a length, and a transverse cross-sectional configuration, wherein said interstitial spaces of one said particle will accept at least one extremity of an adjacent said particle to facilitate interlocking of adjacent particles in said array; and a third shaped particle comprising a multi-ring structure having at least four curved projections wherein said projections provide for interstitial spaces between adjacent said projections, and wherein said projections facilitate interlocking of adjacent particles in said array; and applying said combination to a bone deficiency.

In an embodiment of the present invention, there is a kit for the treatment of a bone deficiency comprising multiple shaped particles, wherein the particles are comprised of bone material and are selected from the group consisting of a first shaped particle comprising a center portion and at least four tapered extremities projecting from said center portion wherein said projections provide for interstitial spaces between adjacent extremities, each extremity having a base attached at said center portion, an opposite point, a length, and a circular transverse cross-sectional configuration, wherein said interstitial spaces of one said particle will accept at least one extremity of an adjacent said particle to facilitate interlocking of adjacent particles in said array of shaped particles; a second shaped particle comprising a center portion, at least two noncurved extremities, and at least three curved extremities projecting from said center portion wherein said projections provide for interstitial spaces between adjacent extremities, each extremity having a base attached at said center portion, an opposite point, a length, and a transverse cross-sectional configuration, wherein said interstitial spaces of one said particle will accept at least one extremity of an adjacent said particle to facilitate interlocking of adjacent particles in said array; and a third shaped particle comprising a multi-ring structure having at least four curved projections wherein said projections provide for interstitial spaces between adjacent said projections, and wherein said projections facilitate interlocking of adjacent particles in said array. In a specific embodiment, the kit comprises a suspension material. In another specific embodiment, the kit further comprises a biological agent. In another specific embodiment, the bone material is allograft material. In another specific embodiment, the kit further comprises a clotting factor composition. In a specific embodiment, the clotting factor composition comprises fibrinogen, thrombin, Factor XIII, or a combination thereof.

In an embodiment of the present invention, there is a shaped particle for use in treating a bone deficiency wherein said particle is comprised of bone material and is shaped for use in an array of particles interlocked with one another, comprising: a center portion; at least two noncurved extremities; and at least three curved extremities projecting from said center portion wherein said projections provide for interstitial spaces between adjacent extremities, each extremity having a base attached at said center portion, an opposite point, a length, and a transverse cross-sectional configuration, wherein said interstitial spaces of one said particle will accept at least one extremity of an adjacent said particle to facilitate interlocking of adjacent particles in said array.

In an embodiment of the present invention, there is a particle manufactured by a method comprising the step of compressing a granulated bone material into said shape. In a specific embodiment, the material further comprises a processing aid composition. In an additional specific embodiment, the processing aid composition is selected from the group consisting of stearic acid, calcium stearate, magnesium stearate, natural polymer, synthetic polymer, sugar and combinations thereof. In another specific embodiment, the processing aid composition is magnesium stearate or stearic acid. In a specific embodiment, the natural polymer is starch, gelatin, or combinations thereof. In a further specific embodiment, the synthetic polymer is methylcellulose, sodium carboxymethylcellulose, or hydropropylmethylcellulose. In an additional specific embodiment, the sugar is glucose or glycerol. In a further specific embodiment, the particle further comprises a biological agent. In a specific embodiment, the biological agent is added to said material prior to said compaction step. In another specific embodiment, the biological agent is added to said bone graft substitute subsequent to said compressing step.

In a specific embodiment, the granulated bone material constituents are less than about 10 millimeters in diameter. In another specific embodiment, the granulated bone material constituents are less than about 250 μm in diameter. In a further specific embodiment, the granulated bone material constituents are in a range of about 50 to 180 microns.

In another embodiment of the present invention, there is a method of manufacturing the particle of claim 1, comprising the steps of obtaining a bone material; processing said material to produce a granulated bone material; and subjecting said granulated bone material to a powder compaction process.

In a specific embodiment, the powder compaction process utilizes a withdrawal press, wherein said press comprises a stationary lower punch; a moveable die; a moveable upper punch; and a moveable lower punch, wherein said stationary lower punch is contained within said moveable lower punch. In a specific embodiment, the powder compaction process utilizes a withdrawal press, wherein said press comprises a stationary lower punch; a moveable lower punch, wherein said stationary lower punch is contained within said moveable lower punch; a stationary upper punch; a moveable upper punch, wherein said stationary upper punch is contained within said moveable lower punch; and a moveable die.

In a further specific embodiment, the method further comprises the steps of providing a stationary lower punch and a moveable lower punch which is vertically moveable about the stationary lower punch, a moveable die having at least one cavity and positionable generally above the stationary lower punch, and a moveable upper punch; introducing the granulated bone material into the cavity; positioning the moveable die generally above the stationary lower punch; moving the moveable upper punch to pressably contact the material in opposition to the moveable lower punch and stationary lower punch; and moving the moveable lower punch to pressably contact the material in opposition to the moveable upper punch, whereby said moving steps form the material into the shaped bone graft substitute. In a specific embodiment, the steps of moving the upper and lower punches effect a substantially uniform distribution of pressure within said material. In another specific embodiment, at least one of the moving steps applies a force to the material in a range of about 0.2 to about 5 tons. In a further specific embodiment, at least one of the moving steps applies a force to the material in a range of about 0.2 to about 2 tons. In an additional specific embodiment, at least one of the moving steps applies a force to the material in a range of about 0.5 to about 1 ton. In another specific embodiment, the moving step of the moveable lower punch to the material is subsequent to the moving step of the moveable upper punch to the material.

In an additional embodiment of the present invention, there is a method of manufacturing a shaped particle as described herein from granulated bone material, said method comprising the steps of introducing an amount of the granulated bone material into the cavity; providing a lower punch assembly, an upper punch assembly, and a moveable die positionable generally above the lower punch assembly; positioning the moveable die generally above the lower punch assembly; moving the lower punch assembly in opposition to the moveable upper punch to pressably contact the material; moving the upper punch assembly in opposition to the moveable lower punch to pressably contact the material, whereby said moving steps form the material into the shaped bone graft substitute. In a specific embodiment, the lower punch assembly is comprised of at least one of a stationary lower punch and a moveable lower punch vertically moveable about the stationary lower punch. In another specific embodiment, the upper punch assembly is comprised of at least one of a stationary upper punch and a moveable upper punch vertically moveable about the stationary upper punch.

In another embodiment of the present invention, there is an apparatus for manufacturing a shaped particle as described herein from granulated bone material, said apparatus comprising a stationary lower punch having a top surface; a moveable lower punch vertically moveable about the stationary lower punch and having a top surface; a moveable die having at least one cavity and positionable generally above the stationary lower punch; and a moveable upper punch, such that said moveable upper punch moves in opposition to said moveable lower punch to pressably contact the material contained within the cavity, whereupon following pressably contacting the material by the moveable lower punch the top surface height of the lower moveable punch is above the top surface height of the stationary lower punch.

In an additional embodiment of the present invention, there is a method for manufacturing a bone graft substitute from granulated bone material, said method comprising the steps of providing a first punch assembly having a first contact surface configured to effect a relief profile onto a first surface of the granulated bone material; a second punch assembly having a second contact surface; and a moveable die having at least one cavity; introducing the bone material into the cavity; positioning the moveable die generally in alignment with the first punch assembly; moving at least a portion of the first punch assembly to pressably contact the material in opposition to the second punch assembly to effect the desired relief profile on the first surface thereof; and moving at least a portion of the second punch assembly to pressably contact the material in opposition to the first punch assembly, whereby said moving steps form the material into the shaped bone graft substitute.

In an additional embodiment of the present invention, there is a method for manufacturing a shaped particle as described herein from demineralized bone matrix material, said method comprising the steps of providing a first punch assembly having a first contact surface configured to effect a relief profile onto a first surface of the demineralized bone matrix material; a second punch assembly having a second contact surface; and a moveable die having at least one cavity; introducing the demineralized bone matrix material into the cavity; positioning the moveable die generally in alignment with the first punch assembly; moving at least a portion of the first punch assembly to pressably contact the material in opposition to the second punch assembly to effect the desired relief profile on the first surface thereof; and moving at least a portion of the second punch assembly to pressably contact the material in opposition to the first punch assembly, whereby said moving steps form the material into the shaped bone graft substitute.

In a specific embodiment, the contact surface area of the first punch assembly is generally equivalent to a contact surface area of the second punch assembly such that the moving steps apply a substantially uniform pressure distribution to the material. In another specific embodiment, the first punch assembly includes a stationary punch and a moveable punch, such that the steps of moving the first punch assembly includes moving the moveable punch to pressably contact the material. In a specific embodiment, the second punch assembly includes a stationary punch and a moveable punch, such that the steps of moving the first punch assembly includes moving the moveable punch to pressably contact the material.

In another embodiment of the present invention, there is an apparatus for manufacturing a shaped particle as described herein from a granulated bone material, said apparatus comprising a first punch assembly having a first contact surface having a profile configured to effect a relief profile onto a surface of the bone material; a second punch assembly having a second contact surface, the second contact surface positioned in general alignment with the first contact surface; and a moveable die having at least one cavity, the moveable die being positionable generally in between the first and second punch assemblies.

Other and further objects, features and advantages would be apparent and eventually more readily understood by reading the following specification and by reference to the company drawing forming a part thereof, or any examples of the presently preferred embodiments of the invention are given for the purpose of the disclosure.

### BRIEF DESCRIPTION OF THE DRAWINGS

For a more complete understanding of the present invention, reference is now made to the following descriptions taken in conjunction with the accompanying drawing, in which:

FIG. 1 is a drawing of a preferred six-armed shaped particle of the invention.

FIG. 2 is a drawing of an array of interlocked six-armed shaped particles of the invention.

FIG. 3A through FIG. 3D are drawings of a five-armed shaped particle of the invention FIG. 3A is a top view of the particle. FIG. 3B is a view of the particle from an elevated side reference. FIG. 3C is a front view of the particle. FIG. 3D is a right view of the particle.

FIG. 4A through 4D are drawings of a six-armed shaped particle of the invention having flat tips. FIG. 4A is a top view of the particle. FIG. 4B is a view of the particle from an elevated side reference. FIG. 4C is a front view of the particle. FIG. 4D is a right view of the particle.

FIG. 5A through 5D are drawings of a six-armed shaped particle of the invention having rounded tips. FIG. 5A is a top view of the particle. FIG. 5B is a view of the particle from an elevated side reference. FIG. 5C is a front view of the particle. FIG. 5D is a right view of the particle.

FIGS. 6A through 6D are drawings of a shaped particle of the invention having an interlocked ring structure. FIG. 6A is a top view of the particle. FIG. 6B is a view of the particle from an elevated side reference. FIG. 6C is a front view of the particle. FIG. 6D is a right view of the particle.

FIGS. 7A through 7D are drawings of different views of a six-armed shaped particle of the invention having a propeller-like structure.

FIG. 8A through FIG. 8D are drawings of a six-armed shaped particle of the invention FIG. 8A is a top view of the particle. FIG. 8B is a view of the particle from an elevated side reference. FIG. 8C is a front view of the particle. FIG. 8D is a right view of the particle.

FIG. 9 shows detailed shape characteristics of an embodiment of the JAX® particle.

FIG. 10 illustrates additional shape characteristics of an embodiment of the JAX® particle.

FIG. 11 shows further shape characteristics of an embodiment of the JAX® particle.

FIG. 12 demonstrates a press configuration used to powder compact JAX® (left) and die and punches (right).

FIG. 13 illustrates a schematic highlighting the differences between (a) conventional tableting and (b, c) the powder compaction used in the novel application to make bone graft substitutes.

FIG. 14 illustrates powder compaction of a jack shape, wherein (a) is filling of a die cavity, (b) is pressably contacting/compacting the material, and (c) is ejection of the product.

FIG. 15 shows powder-compacted JAX® manufactured with HDBM (batch #ALLOJAX100-b).

FIG. 16 depicts scanning electron microscopy (SEM) micrographs of HDBM granules: batch#-a (left), batch#-b (right).

FIG. 17 illustrates an example of a shelf die (cross-section) which is used in an embodiment of manufacturing of the shaped particle comprised of bone material.

FIG. 18 shows powder-compacted tablets made of 100% HDBM, 90% HDBM+10% calcium sulfate, 50% HDBM+50% calcium sulfate and 90% HCC+10% calcium sulfate.

### DESCRIPTION OF THE INVENTION

I. Definitions

The term “allograft bone material” as used herein is defined as bone tissue that is harvested from another individual of the same species. Allograft tissue may be used in its native state or modified to address the needs of a wide variety of orthopaedic procedures. The vast majority of allograft bone tissue is derived from deceased donors. Bone is about 70% mineral by weight. The remaining 30% is collagen and non collagenous proteins (including bone morphogenic proteins, BMPs). Allograft bone that has been cleaned and prepared for grafting provides a support matrix to conduct bone growth, but is not able to release factors that induce the patient's biology to form bone cells and create new bone tissue. In a preferred embodiment, the allograft is cleaned, sanitized, and inactivated for viral transmission.

The term “biological agent” as used herein is defined as an entity which is added to the bone graft substitute to effect a therapeutic end, such as facilitation of bone ingrowth, prevention of disease, administration of pain relief chemicals, administration of drugs, and the like. Examples of biological agents include antibiotics, growth factors, fibrin, bone morphogenetic factors, angiogenic factors, bone growth agents, chemotherapeutics, pain killers, bisphosphonates, strontium salt, fluoride salt, magnesium salt, and sodium salt.

The term “bone deficiency” as used herein is defined as a bone defect such as a break, fracture, void, diseased bone, loss of bone, brittle bone or weak bone, injury, disease or degeneration. Such a defect may be the result of disease, surgical intervention, deformity or trauma. The degeneration may be as a result of progressive aging. Diseased bone could be the result of bone diseases such as osteoporosis, Paget's disease, fibrous dysplasia, osteodystrophia, periodontal disease, osteopenia, osteopetrosis, primary hyperparathyroidism, hypophosphatasia, fibrous dysplasia, osteogenesis imperfecta, myeloma bone disease and bone malignancy. The bone deficiency may be due to a disease or condition, such as a disease that indirectly adversely affects bone. Furthermore, the bone malignancy being treated may be of a primary bone malignancy or may be metastatic, originating from another tissue or part of the body.

The term “bone graft substitute (BGS)” as used herein is defined as an entity for filling spaces in a bone tissue. In a preferred embodiment, the BGS is a shaped particle. In specific embodiments, the BGS as used herein is a jack, such as a JAX®.

In specific embodiments, the particle is shaped for use in an array of particles interlocked with one another, and comprises a center portion; and at least four tapered extremities projecting from said center portion wherein the projections provide for interstitial spaces between adjacent extremities, each extremity having a base attached at the center portion, an opposite point, a length, and a circular transverse cross-sectional configuration, wherein the interstitial spaces of one the particle will accept at least one extremity of an adjacent the particle to facilitate interlocking of adjacent particles in the array.

In other specific embodiments, the particle is shaped for use in an array of particles interlocked with one another, and comprises a center portion, at least two noncurved extremities, and at least three curved extremities projecting from the center portion wherein the projections provide for interstitial spaces between adjacent extremities, each extremity having a base attached at the center portion, an opposite point, a length, and a transverse cross-sectional configuration, wherein the interstitial spaces of one the particle will accept at least one extremity of an adjacent the particle to facilitate interlocking of adjacent particles in the array.

In other specific embodiments, the particle is shaped for use in an array of particles interlocked with one another, and comprises a multi-ring structure having at least four curved projections wherein the projections provide for interstitial spaces between adjacent the projections, and wherein the projections facilitate interlocking of adjacent particles in the array.

In a preferred embodiment a material for the bone grafting system of the present invention is a bone material. In a preferred embodiment, the bone material is a demineralized bone material.

In a specific embodiment the shaped BGS particle of the present invention is colored to make it more visible. In another specific embodiment differently shaped BGS particles of the present invention are denoted with different colors for better differentiation of the particles. In another specific embodiment, the particles are coated or have contained within them an agent such as green fluorescent protein or blue fluorescent protein to make them fluorescent and therefore more visible.

For the JAX® embodiment, the circular cross-section of the extremities, or arms, of the shaped particle of the invention is beneficial for strength purposes, because an equivalent response to loading will occur regardless of the application of the load around the circumference. In contrast, an oval shape as is utilized in commercially available products and in U.S. Pat. No. 5,676,700 has reduced resistance to loading when the loading is applied in the direction of the axis of the shorter width of the oval compared to the axis of the longer width of the oval.

The term “bone material” as used herein refers to material derived from the bone tissue of an organism. The bone may come from a human or another organism. In a specific embodiment, the bone material is allograft material. In another specific embodiment, the bone material is demineralized bone material.

The term “demineralized bone material” as used herein is defined as a bone material which has been treated for removal of minerals within the bone. Examples of demineralization processes known in the art include BioCleanse (Regeneration Technologies, Inc.) or D-MIN (Osteotech, Inc.). In a specific embodiment, the allograft material is subjected to a series of thermal (freezing), irradiation, physical, aseptic, and/or chemical (acid soak) processes known in the art. The latter (acid soak) typically consists of a proprietary permeation treatment to dissolve the minerals contained in the bone. This series of processes combine both demineralization and anti-viral activity. A skilled artisan recognizes that the actions of bone morphogenic proteins (BMPs) are inactivated by the mineral matrix of the bone. Demineralized bone material (DBM) is created from a process that removes the mineral content and allows the bone morphogenic proteins to operate. In addition to removing bone mineral, the processes used to produce DBM also have viral inactivating properties, providing an added assurance of safety for DBM products.

The term “granulated bone material” as used herein is defined as a composition comprising particles such as grains, granules, powder, and the like. The particles are preferably comprised of a substance or substances which are amenable for bone growth, bone repair, bone augmentation, and the like. In a specific embodiment, the granulated bone material further comprises a processing aid composition. In a specific embodiment, the mixture is primarily comprised of finely dispersed solid particles. In another specific embodiment, one must view the particles under a microscope to differentiate one particle from another. The powder can be comprised in a granular form, such as the singular particles seen in sugar, or it can be in a granulated form, as in an agglomerate of particles. In a preferred embodiment, it is not a chip. In a specific embodiment, at least the majority of the particles in the mixture are less than about 10 mm in diameter. In a more preferred embodiment, the majority of particles in the mixture are less than about 250 microns in diameter. In a most preferred embodiment, the majority of the particles in the mixture are between about 50 and about 180 microns in diameter.

The term “suspension material” as used herein refers to any material that suspends the shaped particles of the invention for easier application to a bone deficiency. A suspension material may be used as an additional component of a system for a bone graft substitute to treat bone deficiency. The suspension material may be a liquid, putty, dough or gel phase component and may be mixed with the shaped particles described above at the time of use or come as a pre-packaged system. The suspension material could serve two potential functions: 1) to act as a binder to improve handling by forming a putty-like material which is shapeable, and/or 2) to act as a biological tool to assist in the healing through the addition of infection control, bone growth, or other healing or biological agents. The suspension material can provide standard suspension of particles within a material or it may provide adhering of particles or connecting of particles in a manner wherein the material is smaller in volume in an array than the volume of the particles themselves.

The suspension material can either be setting or non-setting in response to time, temperature, presence of body fluid or other external stimuli which might supply energy, such as ultraviolet radiation, magnetic radiation, electromotive force (EMF), radiowaves, or ultrasound. In one embodiment the suspension material will degrade once implanted. Ideally, it would be derived from naturally occurring substances such as carbohydrates, starches or glycerin. It should have a sufficient viscosity as to help the granules adhere to each other to improve intraoperative handling. In the ceramic particle embodiments, coating the calcium with this type of substance may also decrease their affinity to stick to soft tissue, making it easier to remove unwanted pieces from the application site. Fibrinogen/thrombin/Factor XIII combinations may also provide a liquid or gel of appropriate viscosity to use as a binder. The liquid may also be a synthetic material such as calcium sulfate (plaster of Paris) that would set in situ. In another embodiment, this binder could act as a carrier for a variety of agents including but not limited to growth factors, bone morphogenic proteins, fibrinogen/thrombin, antibiotics or some other therapeutic agent.

In a specific embodiment the suspension material is blood, bone marrow, autograft material, or allograft material. These materials are preferentially derived from the patient with the bone deficiency being treated. Alternatively, they are derived from a donor and preferable are free from being the source of disease transmission.

An example of a suspension material is a mixing gel which can be mixed with the synthetic or natural products (autograft or allograft) of choice by the clinician to produce a ‘paste’ for application to a bone deficiency such as bone void filling. The suspension material must have the appropriate viscosity and tackiness to agglomerate the particles for easy application to the graft site. Once agglomerated, the paste could be manipulated by hand or be transported by use of a tool such as a scoop, spoon or syringe to the defect site.

The suspension material can also reduce the preferential sticking to soft tissue. This adhesion to soft tissue may be caused by a number of factors. For example, many commercially available products have rough surfaces that may mechanically adhere to soft tissues. A suspension material can minimize the effect. The suspension material can alter the surface chemistry of the particle, thus reducing the particles' affinity for proteins. The suspension material also fills in rough features, thereby reducing the particles' ability to mechanically adhere to the tissue.

The suspension material of the present invention may be comprised of biocompatible polymers, and in a specific embodiment the polymers are bioresorbable. The polymers must be graftable into an animal without causing unacceptable side effects. The polymers may be homopolymers or copolymers and are preferably amorphous. A specific example is polymers in which the units are derived from hydroxy carboxylic acids, which are polyesters. Another example is poly(lactic acids) which may originate from the polymerization of mixtures of L- and D-lactides in proportions such that the poly(lactic acids) are amorphous. Another example is copolymers consisting of units derived from lactic and glycolic acids.

A biocompatible polymer may or may not be degradable, depending on the proposed use. Degradable polymers which are nontoxic and implantable into organisms such as humans are preferable, and examples include polyglycolic acid or polylactic acid. Other materials which may be useful based on their biocompatibility and the ability to alter their viscosity and tackiness to prove useful in this invention include: polyvinylpyrolidone, chitosin, glycerol, carboxymethylcellulose, methylcellulose, carrageenan, hyaluronic acid, collagen-hydroxyapatite-hyaluronic acid composite, alginate, dextrose, starches, cellulose gums or combinations of any of the above listed items. A skilled artisan is aware that collagen or a derivative of collagen is preferably treated prior to use in the invention so as not to be immunoreactive, or alternatively a recombinant form of collagen may be used.

A binder is a material that aids in the agglomeration of the particles due to the tackiness of the binder both in a cohesive (with itself) and adhesive (with the particles) nature. The final construct (binder plus particles) still has flexibility and pliability so that it can fill a defect completely. It is possible that plaster of Paris or a settable calcium phosphate cement system may be used as a binder which will still ultimately set to a firm construct. This would provide an improvement in the immediate structural strength under a loading pattern that is predominately compression. So, therefore, a binder may or may not harden. In a preferred embodiment the binder hardens.

Examples of appropriate physiological materials which may be included in the suspension material are saline, various starches, hydrogels, polyvinylpyrrolidines, other polymeric materials, polysaccharides, organic oils or fluids, all of which are well known and utilized in the art. Materials that are biologically compatible, i.e., cause minimal tissue reaction and are removed or metabolized without cytotoxicity, are preferred. Biologically compatible saccharides such as glucose or aqueous solutions of starch may be used. Certain fats may also be used. In this connection, highly compatible materials include esters of hyaluronic acids such as ethyl hyaluronate and polyvinylpyrrolidone (PVP). PVP normally has the general empirical formula [CHCH₂)₂N(CH₂)₃CO]ₙ wherein n equal 25-500, a form otherwise known as Plasdone® (trademark of GAF Corporation, New York, N.Y.). Another biocompatible material is a patient's own plasma. Blood may be withdrawn from the patient, centrifuged to remove cells (or not) and mixed with appropriate volume of particles and the mixture applied in the desired locations.

In a preferred embodiment the suspension material is comprised of the following: carboxymethylcellulose (maximum of 3 weight percent); glycerol USP (maximum of 20 weight percent); and purified water USP (maximum of 88.75 weight percent). The advantages to utilizing the suspension material of the invention which are improvements over currently available products derived from human tissue include: improved handling; lower cost; no risk of disease; easier storage; longer shelf life; ease of discarding any excess material; compatibility with all known synthetics; and unlimited supply.

The term “tapered” as used herein is defined as referring to an extremity of a shaped particle wherein the width of one end of the extremity is different in size from the width of another end of the extremity. That is, the tapering of the extremity may be outward away from the center of the particle or may be inward toward the center of the particle.

The term “JAX®” as used herein is defined as a bone graft substitute particle which generally has the shape of a toy jack. In a specific embodiment, it is a three-dimensional six-armed star-like shape.

The term “powder compaction” as used herein is defined as the process wherein a granulated bone material, such as a powder, is compressed into a desired shape. In a preferred embodiment, the powder is demineralized bone matrix. In another preferred embodiment, the powder particles are less than about 10 mm, more preferably less than about 250 μm, and most preferably between about 50 and 180 microns in diameter.

The term “pressably contact” as used herein is defined as the touching of a material using pressure upon the material. In a specific embodiment, pressably contacting the material results in compaction of the material, such as in compaction of a granulated bone material, for example a powder.

The term “process” as used herein is defined as pulverize, grind, granulate, crush, mill, mash, chop up, or pound a starting material into smaller constituents. In a specific embodiment, the starting material is reduced to powder or dust.

The term “processing aid composition” as used herein is defined as a composition utilized for facilitating compaction of a powder and release of a compacted powdered product from a die. Specific examples include stearic acid, magnesium stearate, calcium stearate, natural polymer, synthetic polymer, sugar and combinations thereof. In a specific embodiment, the natural polymer is starch, gelatin, or combinations thereof. In another specific embodiment, the synthetic polymer is methylcellulose, sodium carboxymethylcellulose, or hydropropylmethylcellulose. In an additional specific embodiment, the sugar is glucose or glycerol.

The term “relief profile” as used herein is defined as a contour on a material having projections and indentations that approximate the contour of the surface which imparts the contour, such as a punch.

The term “substantially uniform distribution of pressure” as used herein is defined as an amount of pressure upon a material that is generally consistent in quantity over the surface of the material.

The term “three-dimensional intricate shape” as used herein is defined as a shape having projections and/or at least one surface that has a relief profile.

II. The Present Invention

An object of the present invention is a shaped particle as part of three-dimensional interlocking array of particles to be utilized in bone graft. A skilled artisan is aware that the particles may be utilized with inductive graft in which the graft actively facilitates, either directly or indirectly, bone growth. In addition or alternatively, the particles may be utilized for a conductive graft in which the graft is conducive to bone growth but does not actively or directly facilitate it.

In a specific embodiment, the particle is comprised of a bone material. In a further specific embodiment, the bone material is demineralized bone material, such as fully demineralized, partially demineralized, or a mixture thereof. In another specific embodiment the particles are augmented with a biological agent. The particles will be of an appropriate size such that several individual granules will be used to fill a small void while many can be used to fill larger voids. The three-dimensional structure will allow the granules to fill a volume and interlock with each other. In addition, the particles will be able to interlock with bone already present in the recipient individual. The interlocking will enable the particles to support some mechanical forces while maintaining stability and assist in bone healing. The interlocking feature makes it possible for the particles to resist some shear forces, unlike commercially available products. It will also help to resist migration away from the implant site. The particles will be able to fill odd bone defect shapes and sizes without necessarily needing to carve a larger block to the approximate shape/size. The interlocked particles also provide the ability for the entire implant to behave mechanically more like a single block as compared to current granular products. The shapes would be such that a collection of these particles do not aggregate into a solid, packed volume but instead leave an open, interconnected porosity that is beneficial for bone healing. It is preferred that the shape of the particles and/or the array of the shaped particles allow the engineering or prediction of a specific porosity. For example, the particles can be shaped to have such a design as to allow 40-80% porosity upon agglomeration.

The purpose of having shaped particles is two-fold. First, the capability to interlock provides resistance to shear forces and helps to increase the stability when the graft is packed into a defect. Second, porosity needs to be maintained when the shaped particles are interlocked. It is known in the art that new bone growth can ingress into pores ranging from 100-400 microns in size. The targeted total porosity will range from 20% to 80%, which means that the array of interlocking shaped particles of the invention will retain open spaces of 20-80% of a specific volume of an array. It is important that a graft material provide adequate porosity to allow ingrowth from the host bone. Alternatively, the material must resorb or degrade away to allow for bone replacement. The preferred embodiment is the combination of both of these properties.

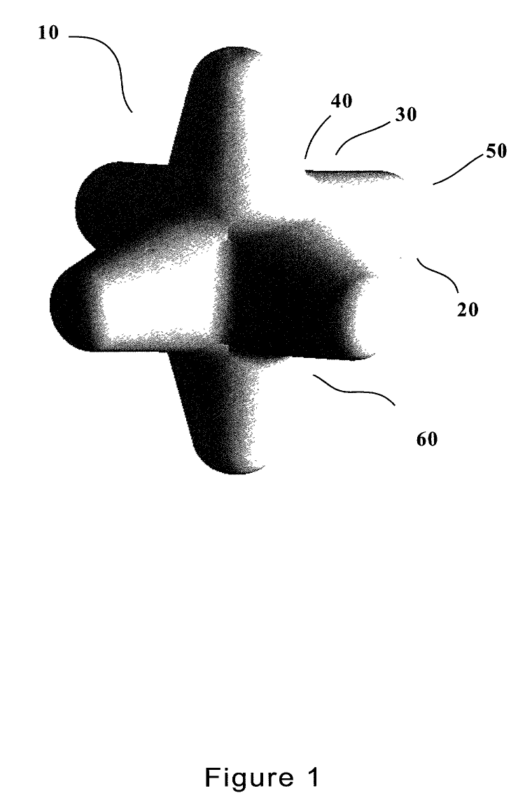

The tapering of the extremities of the shaped particles improves manufacturability, maximizes the open space between the extremities, and provides greater mechanical stability in, for instance, the preferred shaped particle of FIG. 1. This is due in part because the arms are thicker closer to the central body, which distributes loads over more mass of material.

The shaped particles of the present invention are illustrated in the FIGS. FIG. 1 shows a shaped particle (10) having an extremity (20), and in a preferred embodiment the particle has six extremities. In a preferred embodiment at least three of the extremities are in a common plane. The extremities are tapered outwardly along the length (30) of the extremity so that the base (40) of the extremity is wider than the tip (50) of the extremity. In a preferred embodiment the tip (50) of the extremities are rounded. The particle has an interstitial space (60) between the adjacent extremities (20). In a preferred embodiment the radius of curvature of the tip (50) of an extremity (20) is about 0.5 mm and the radius of curvature of the interstitial space (60) between adjacent extremities is about 0.5 mm. The preferred width of the entire particle is about 3-10 mm, and more preferred 4-8 mm, and most preferred is 6 mm. The preferred width of a base (40) of an extremity (20) is about 1.85 mm, the preferred width of a tip (50) of an extremity is about 1.19 mm, and the preferred length (30) of an extremity (20) is about 3 mm. In a preferred embodiment the angles between any of the adjacent extremities (20) are approximately equal. A skilled artisan is aware that shaped particles may be used which are greater in size than these measurements or smaller in size than these measurements depending on the relevant application and bone deficiency. It is preferred to keep the size of the particle small relative to the wound site so that it will take many particles to fill the defect rather than one.

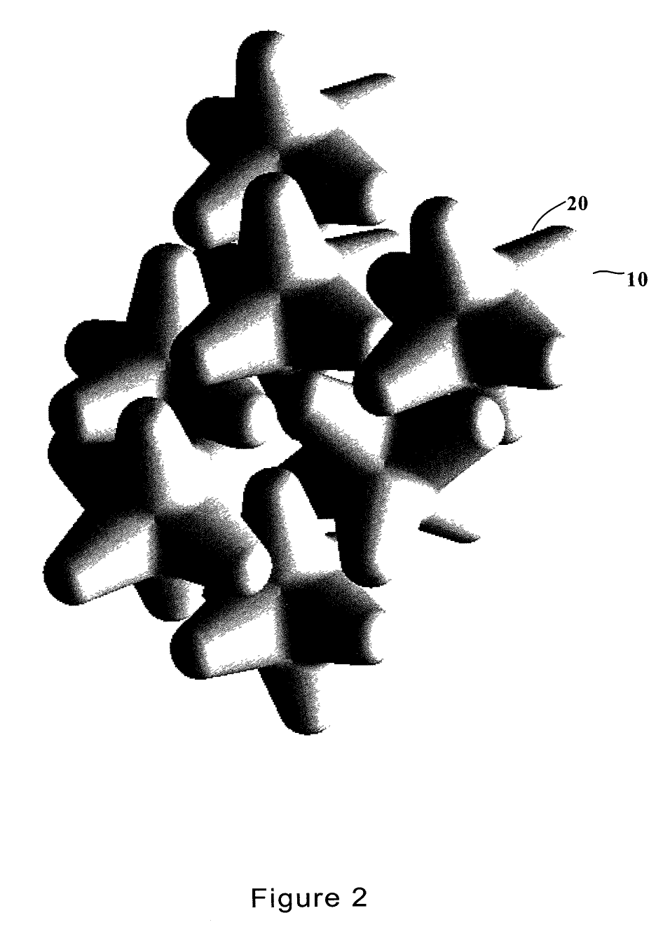

FIG. 2 illustrates an array of shaped particles of the invention wherein the extremities (20) of adjacent particles (10) are interlocked.

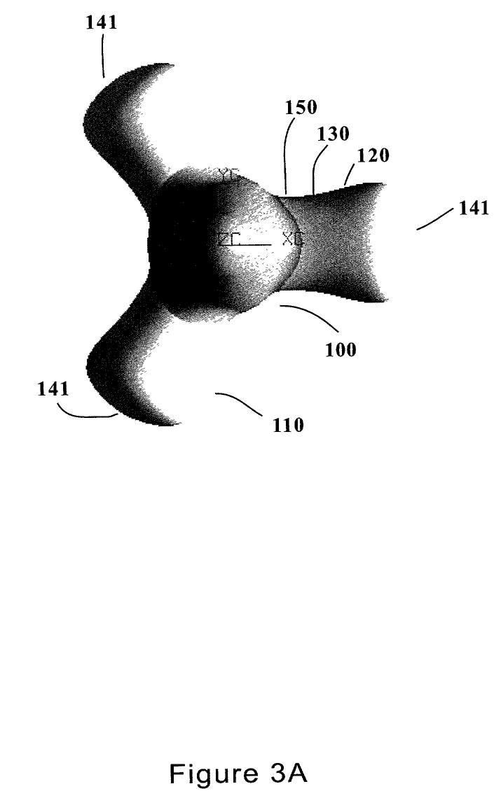

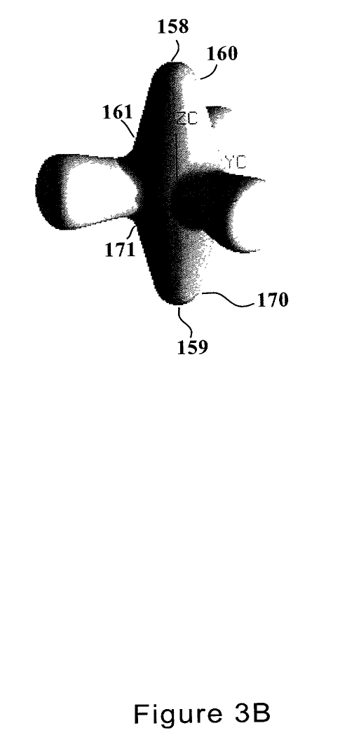

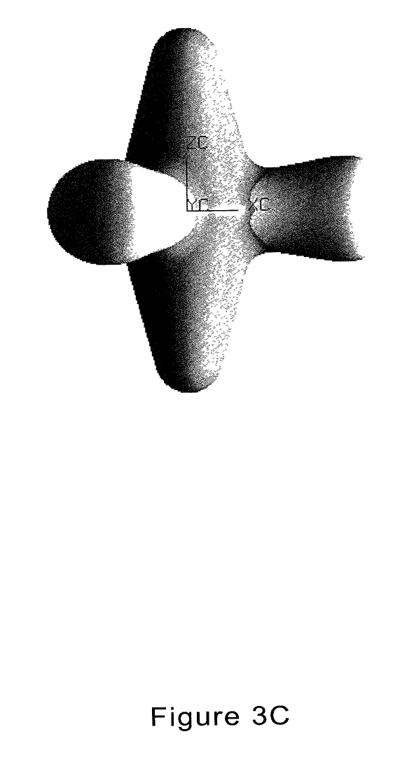

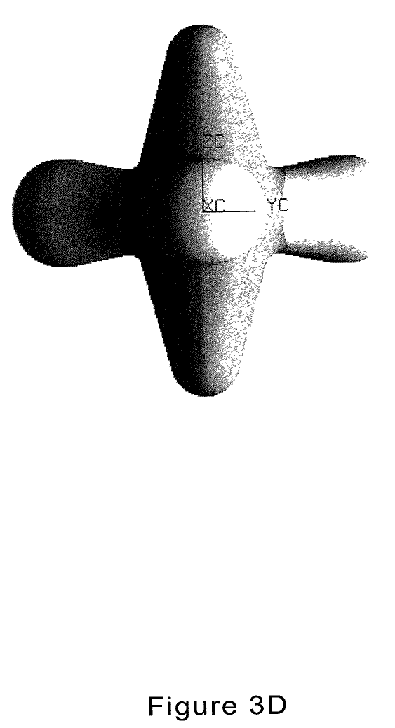

FIGS. 3A through 3D illustrate different views of a specific embodiment wherein a five-armed shaped particle (100) is an object of the invention. In a preferred embodiment of a five-armed shaped particle at least three extremities lie in a plane. An extremity (110) is tapered inwardly along its length (120) wherein the base (130) of the extremity (110) is more narrow in width than the tip (141) of the extremity (110). An interstitial space (150) is present between adjacent extremities. The tips (141) of the extremities (110) are rounded in a specific embodiment. FIGS. 3B through 3D illustrate that in a specific embodiment the tips (158 and 159) of two extremities (160 and 170, respectively) which are situated about 180 degrees from one another are generally more conical in shape than the tips (141) of the extremities (110). The extremities (160 and 170) taper outwardly where the base (161 and 171, respectively) is wider than the tips (158 and 159).

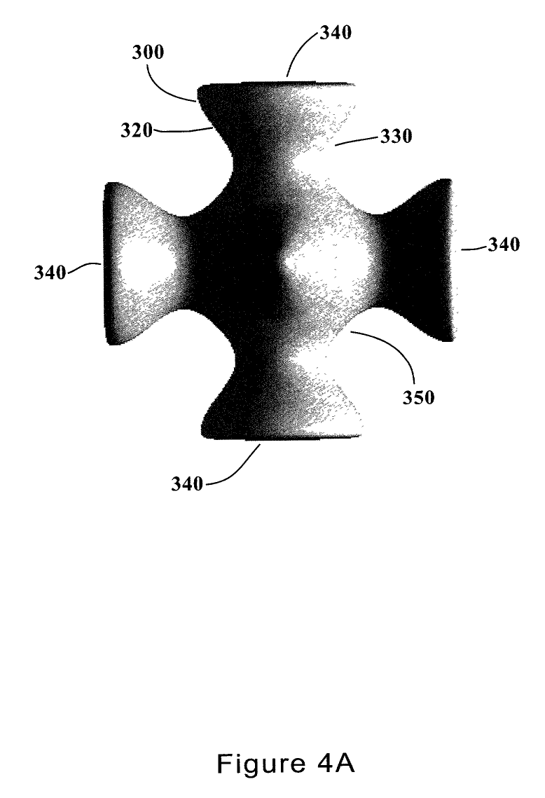

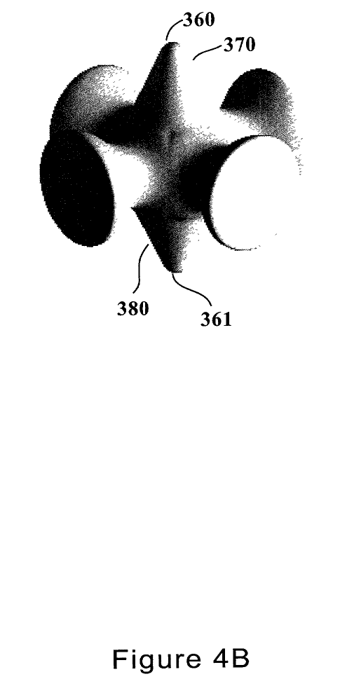

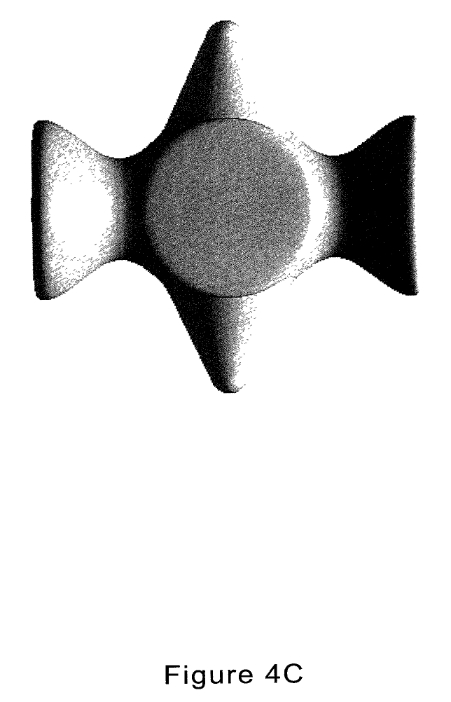

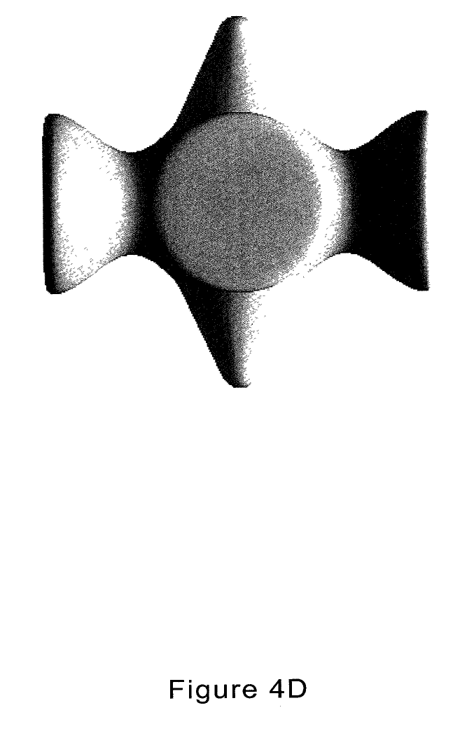

FIGS. 4A through 4D illustrate different views of a specific embodiment wherein a six-armed shaped particle (300) is an object of the invention. In a preferred embodiment at least three extremities lie in a plane. An extremity (310) is tapered inwardly along its length (320) wherein the base (330) of an extremity (310) is more narrow in width than the tip (340) of the extremity (310). An interstitial space (350) is present between adjacent extremities. The tips (340) have a generally flat surface. FIGS. 4B through 4D show the tips (360 and 361) of two extremities (370 and 380, respectively) are generally more conical in shape than the tips (340) of the extremities (310) and are situated about 180 degrees from one another in the particle (300).

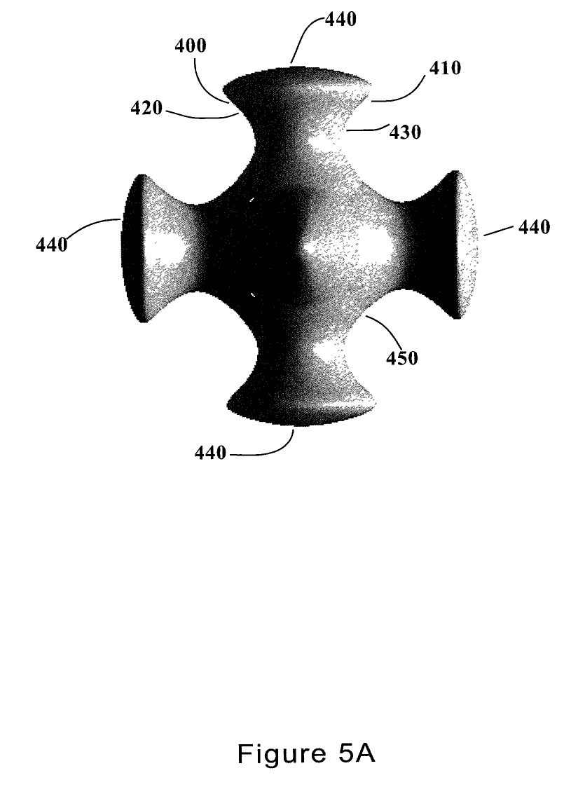

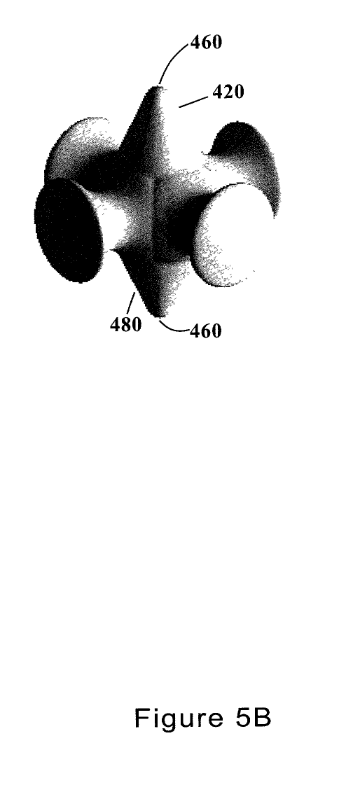

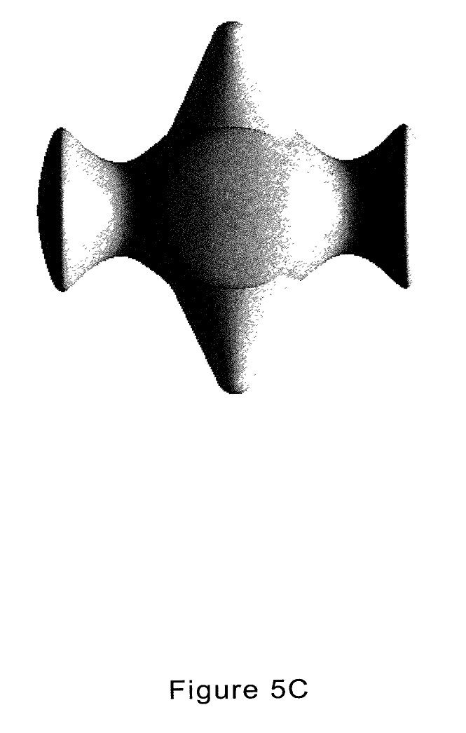

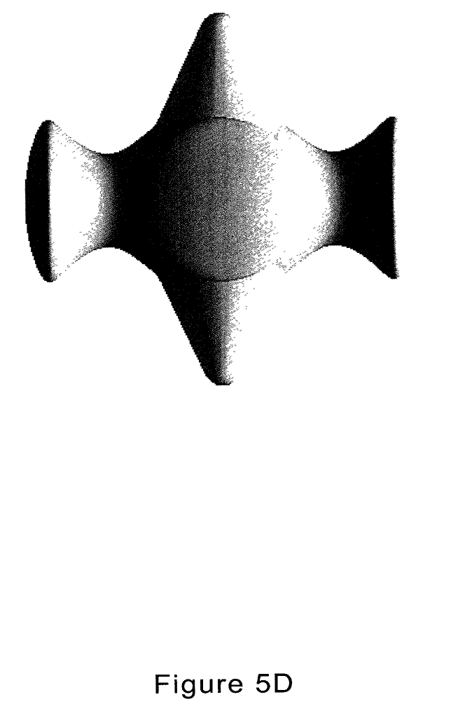

FIGS. 5A through 5D illustrate different views of a specific embodiment wherein a six-armed shaped particle (400) is an object of the invention. In a preferred embodiment at least three extremities lie in a plane. An extremity (410) is tapered inwardly along its length (420) wherein the base (430) of an extremity (410) is more narrow in width than the tip (440) of the extremity (410). An interstitial space (450) is present between adjacent extremities. The tips (440) of the extremities (410) have a generally rounded surface. FIGS. 5B through 5D show the tips (460 and 461) of two extremities (470 and 480 respectively) are generally more conical in shape than the tips (440) and are situated 180 degrees from one another in the particle (400). The tapering inwardly of the extremities (310 and 410) allows these shaped particles to “snap-fit” into an adjacent particle.

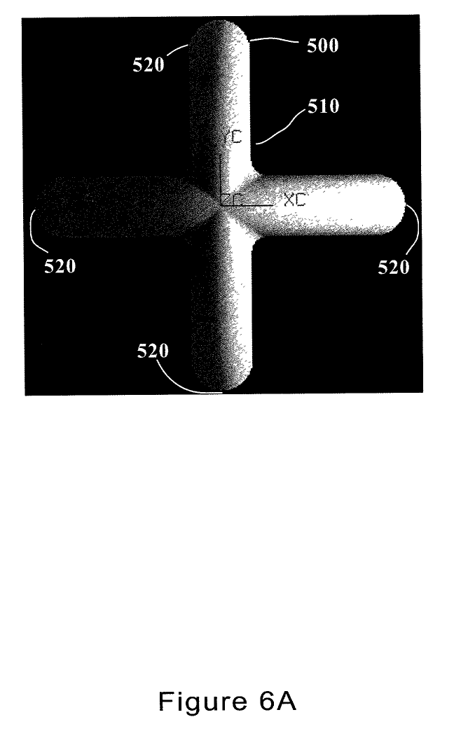

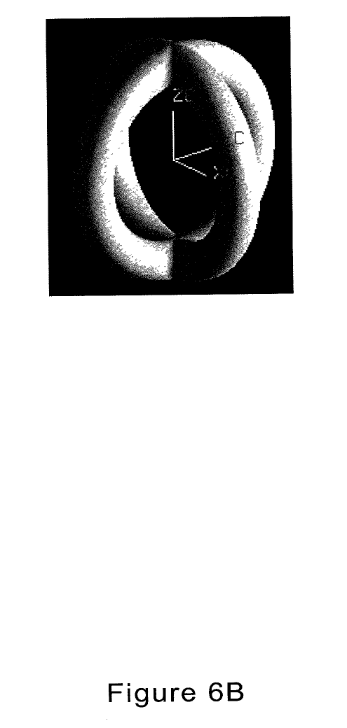

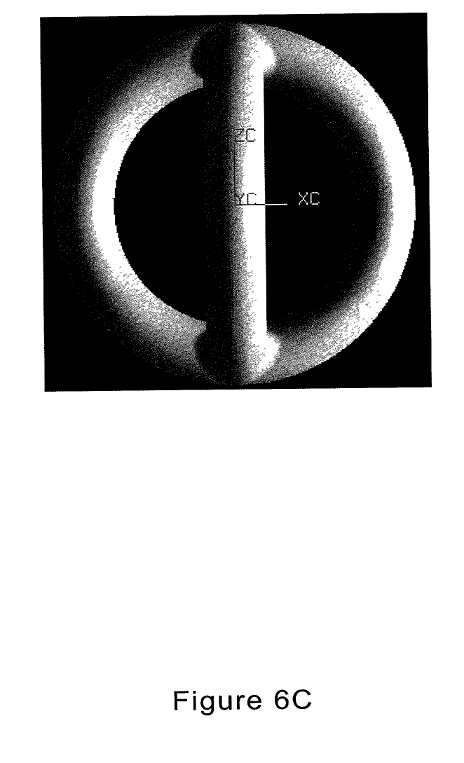

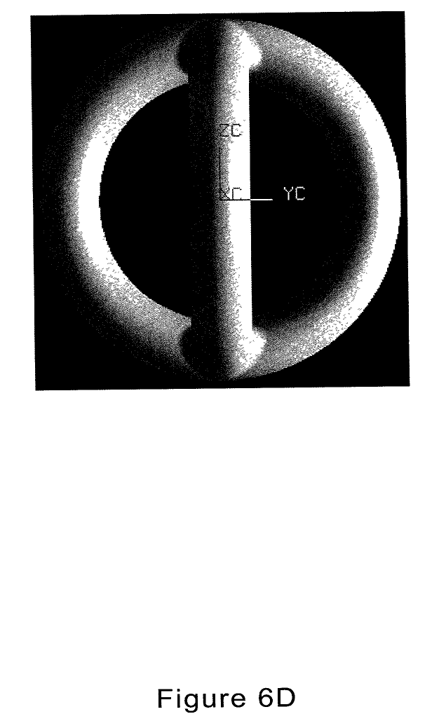

FIGS. 6A through 6D illustrate different views of a specific embodiment of the present invention wherein a shaped particle (500) is similar to two interlocked rings positioned at about 90 degrees from one another. Interstitial spaces (510) allow interlocking of the rings (520), or curved projections, of an adjacent particle. A preferred diameter of the entire particle (500) is about 6 mm, and a preferred diameter of the ring (520) component of the structure is about 1 mm. The maximum number of rings would be such that the surface area of the rings should not be more than 50% of the surface area of the encompassed sphere—otherwise the parts would not interlock or nest with each other. Using this as a starting point, then the diameter of the solid structure of the ring (as an example at about 1 mm) becomes a factor. As that diameter decreases the number of possible rings increases.

In the mathematical relationship between a radius of a “spherical” particle, r, a thickness or diameter of rings, d, and a number of rings, n, a surface area of a sphere is 4πr² and a surface area of the interlocking rings is 2πrdn. The objective is that the surface area of the rings is less than or equal to 50% of the surface area of a sphere. The mathematical relationship can be described as

2πrdn≦0.50(4πr²), or

2πrdn≦2πr², or

dn≦r.

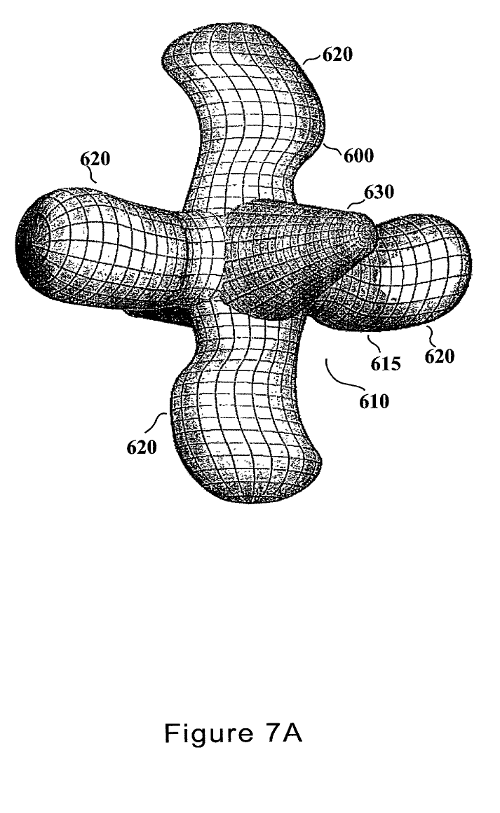

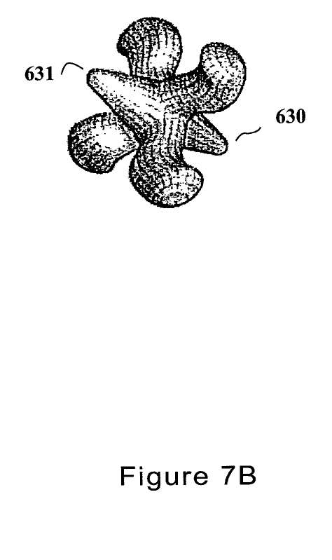

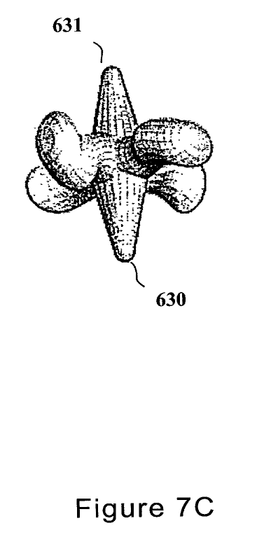

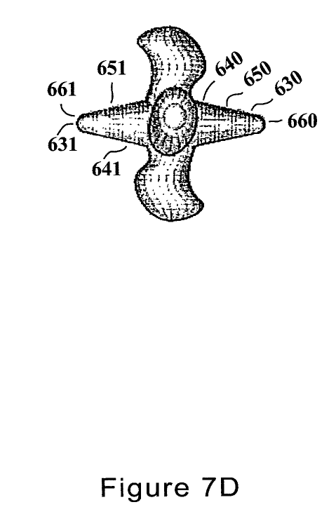

FIGS. 7A through 7D illustrate a specific embodiment of the present invention wherein a shaped particle (600) is similar to a propeller. Interstitial spaces (610) allow interlocking of the extremities (620) of the particle. The length (615) of an extremity (620) is curved generally as in a propeller arm. The composition material of this structure is a ceramic, polymer, bioglass, polymer/ceramic composite, or polymer/glass composite. In a preferred embodiment the structure is relatively compliant in comparison to a ceramic-based structure. A preferred diameter of the entire particle (600) is about 6 mm, and a preferred diameter of the extremities (620) component of the structure is about 1 mm. The extremities (630 and 631), particularly as shown in FIG. 7D, are generally conical in shape, having a wider base (640 and 641, respectively) tapering along the length (650 and 651, respectively) of the extremity to a narrower tip (660 and 661, respectively). The extremities (630 and 631) are positioned about 180 degrees relative to each other.

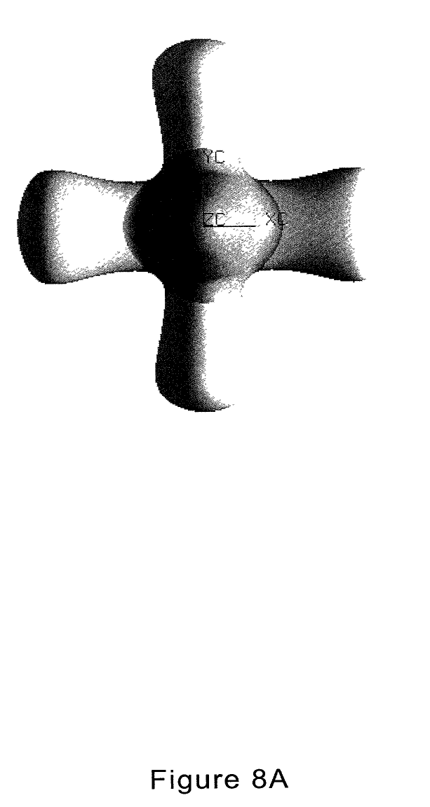

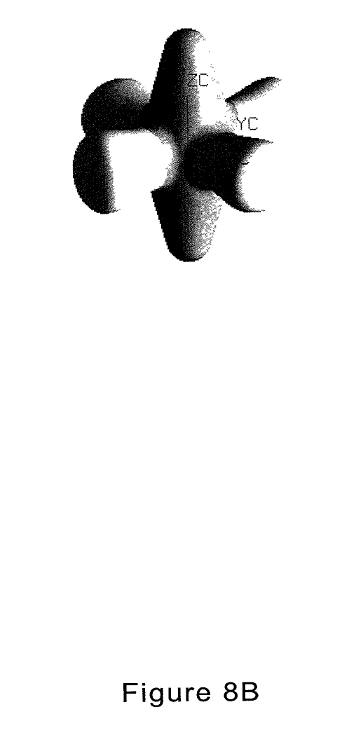

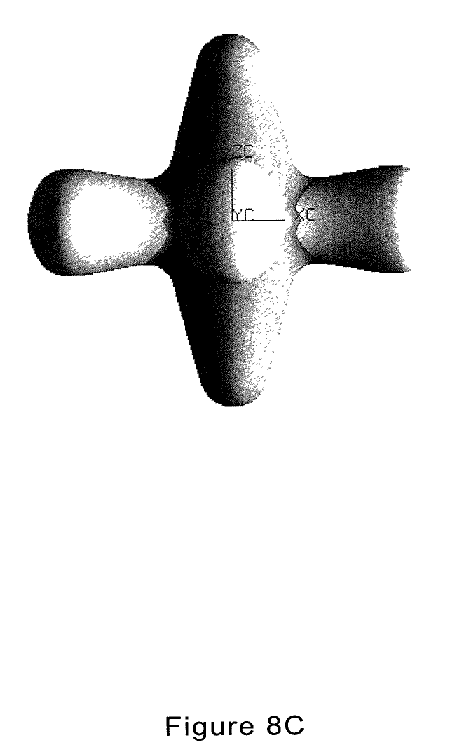

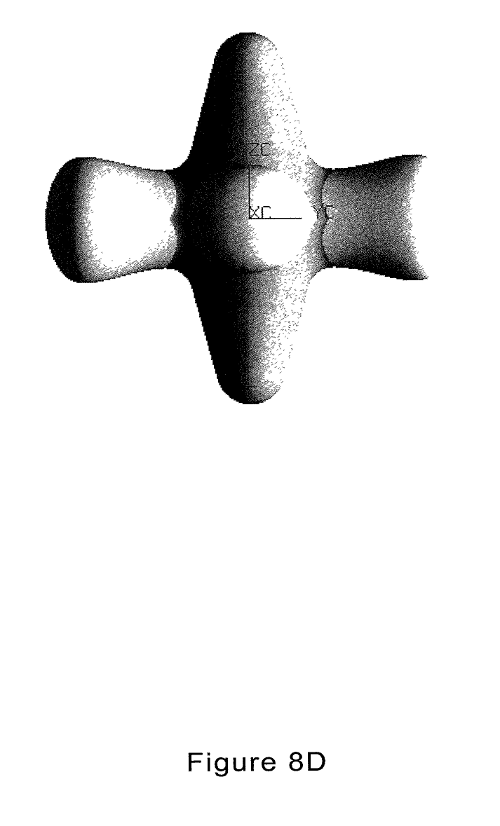

FIGS. 8A through 8D illustrate different views of a specific embodiment wherein a six-armed shaped particle (700) is an object of the invention. In a preferred embodiment of a six-armed shaped particle at least three extremities lie in a plane. An extremity (710) is tapered inwardly along its length (720) wherein the base (730) of the extremity (710) is more narrow in width than the tip (741) of the extremity (710). An interstitial space (750) is present between adjacent extremities. The tips (741) are rounded in a specific embodiment. FIGS. 8B through 8D illustrate that in a specific embodiment the tips (702 and 704) of two extremities (760 and 770, respectively) which are situated about 180 degrees from one another are generally more conical in shape than the tips (741) of the extremities (710). The extremities (760 and 770) taper outwardly where the base (761 and 771, respectively) is wider than the tips (702 and 704, respectively).

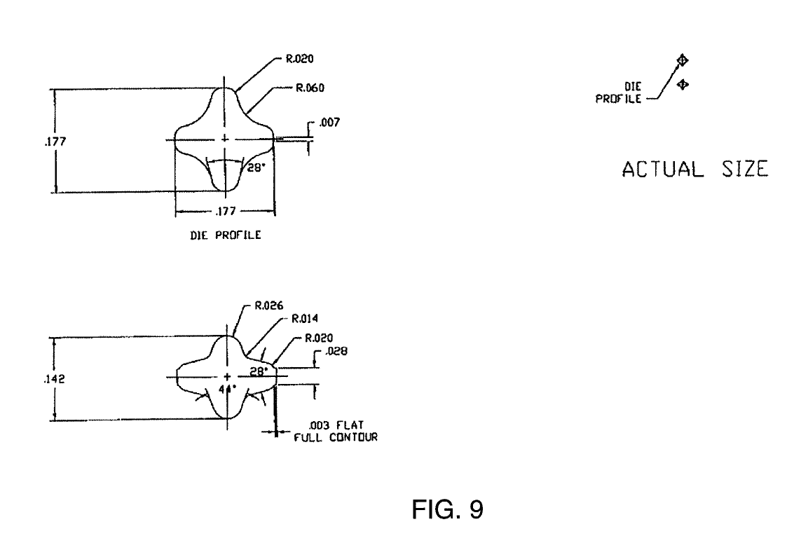

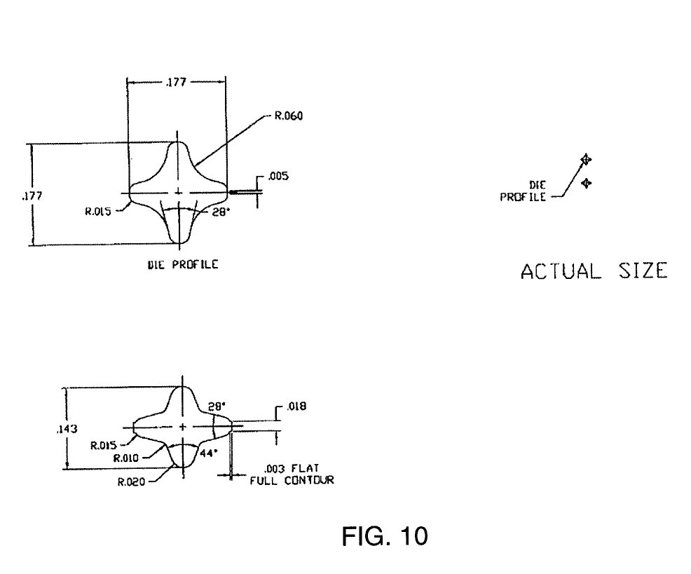

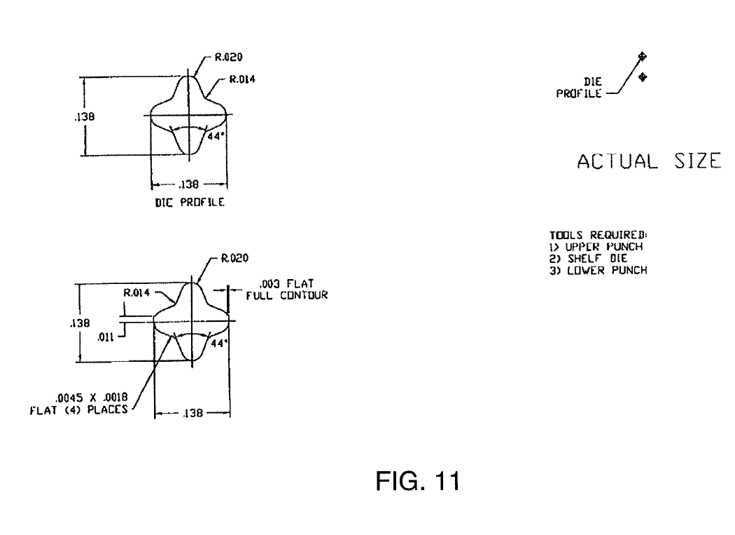

FIGS. 9 through 11 illustrate detailed characteristics of an embodiment of the JAX® particle.

A skilled artisan is aware that the surface to volume ratio of the shaped particle of the present invention has influence upon several factors, including the intended application of the bone graft, which dictates the size of the particle needed and the dissolution rates, strength and manufacturability.

In embodiments of the present invention, a powder compaction process is used to produce a bone graft substitute, such as a JAX® product comprised of DBM. A processing aid is added to facilitate compaction of the DBM powder and release of the product from the die. A biological agent may also be added to the powder prior to compaction or coated onto the generated product after compaction. The present invention is an improvement over presently available products and methods by taking, in a specific embodiment, an allograft powder, as opposed to a chip, and manufacturing a shape from the powder, wherein the shape is used for a bone graft substitute.

The material from which the BGS is manufactured is a granulated or granular bone material powder, such as an allograft material, a synthetic material, a ceramic material, a polymer, or combinations thereof. The allograft material may be processed, such as subjected to a demineralization process, or it may be unprocessed, in which minerals remain intact. The material in any case is preferably cleaned, sanitized, and inactivated for pathogen transmission, such as a virus. The allograft material may be of cortical-cancellous bone or demineralized bone matrix.

In a specific embodiment of the present invention, the bone graft substitute is manufactured with a biological agent, either within the particle, coated on the surface of the particle, or both.

It is preferable for the allograft bone graft substitute embodiment of the present invention to have a granule or shape for easy delivery and scaffold structure. An object of the present invention is providing a BGS that is a shaped particle which may be used as part of a three-dimensional interlocking array of particles. A skilled artisan is aware that the particles may be utilized with inductive graft in which the graft actively facilitates, either directly or indirectly, bone growth. In addition or alternatively, the particles may be utilized for a conductive graft in which the graft is conducive to bone growth but does not actively or directly facilitate it.

The particles will be of an appropriate size such that several individual granules will be used to fill a small void while many can be used to fill larger voids. The three-dimensional structure will allow the granules to fill a volume and, in a specific embodiment, interlock with each other. In another specific embodiment, the particles will be able to interlock with bone. The interlocking will enable the particles to support some mechanical forces while maintaining stability and assist in bone healing. The interlocking feature makes it possible for the particles to resist some shear forces, unlike commercially available products. It will also help to resist migration away from the implant site. The particles will be able to fill odd bone defect shapes and sizes without necessarily needing to carve a larger block to the approximate shape/size. The interlocked particles also provide the ability for the entire implant to behave mechanically more like a single block as compared to current granular products. The shapes would be such that a collection of these particles do not aggregate into a solid, packed volume but instead leave an open, interconnected porosity that is beneficial for bone healing. It is preferred that the shape of the particles and/or the array of the shaped particles allow the engineering or prediction of a specific porosity.

The purpose of having shaped particles is three-fold. First, the capability to interlock provides resistance to shear forces and helps to increase the stability when the graft is packed into a defect. Second, porosity needs to be maintained when the shaped particles are interlocked. It is known in the art that new bone growth can ingress into pores ranging from 100-400 microns in size. The targeted total porosity will range from 20% to 80%, which means that the array of interlocking shaped particles of the invention will retain open spaces of 20-80% of a specific volume of an array. It is important that a graft material provide adequate porosity to allow ingrowth from the host bone. Alternatively, the material must resorb or degrade away to allow for bone replacement. The preferred embodiment is the combination of both of these properties. Third, the shaped particles provide superior handling of BGS product during transfer into the surgical site.

III. Polymeric Shaped Particle

In an alternative object of the present invention, the shaped particles of the invention are of a polymeric phase. The material could be derived from a wide variety of bioabsorbable, biocompatible polymers that will resorb or degrade over time. These polymers could also be ceramic or glass filled in order to boost the osteoconductivity of the polymer alone. The polymers, or composites, also allow control of mechanical properties, such as strength and stiffness, and control of degradation rates. The function of this component is to offer compliance to a bone graft system comprised of this material and the ceramic and suspension material phases described above. In a preferred embodiment the polymeric shaped particles will interlock with a ceramic-based particle, still maintaining a certain volume of the combination that is open and has an interconnected porosity. The polymeric granule also protects the ceramic components from brittle fracture under compaction, acting as a buffer while the system is compressed to fill a bone deficiency. In order to achieve these properties it is envisioned that the polymeric shaped particles will be mostly plastic in their behavior with a small portion of elastic response. This will insure that the polymeric shaped particles will compress without too much rebound, but that they will also serve as buffers between the ceramic granules. It is also conceivable that the polymeric/composite granules may be used without the ceramic granules in some indications where the ability to compact the material is very important, such as in the compaction grafting technique commonly used today in total joint revisions. No current ceramic shaped particle system is suitable for compaction since they would be pulverized by this technique.

In a preferred embodiment the shaped particle of polymer has as the ends of its extremities a bubble shape which may provide a “snap-fit” for adjacent interlocking polymeric shaped particles, such as the particles illustrated in FIGS. 4 and 5.

IV. A Bone Graft System

Together, the components of the invention that provide a bone graft substitute system, including a bone material or ceramic shaped particle, a suspension material, and, in some embodiments, a polymeric shaped particle, will offer the clinician several options when approaching a grafting procedure. The most basic option would be to use the bone material granules alone when the defect is contained and does not have to provide a lot of mechanical or structural support. When the suspension material is added the clinician will be able to work with the granules outside of the bone deficiency site to shape the aggregate. The suspension material may also offer the possibility to introduce infection control or active agents to promote bone healing and growth. In a specific embodiment, bone material shaped particles are utilized with polymeric or ceramic particles and/or the suspension material. The addition of the polymeric shaped particles to the ceramic shaped particles offers the clinician the ability to compress the graft into a deficient site. This would be beneficial when more structural support and stability was required of the implant and might also be more suited to larger volume defects. The system may also include additional allograft material, such as chips, blocks, putties and gels, or in addition or alternatively may include autograft material.

In a specific embodiment the system will include multiple shaped particles wherein the particles are of different shapes. The different shapes which may be included are illustrated in the figures herein or may have variations of these shapes. In addition or alternatively these multiple particles may be comprised of different materials.

As seen from the currently available products, the typical approach to address the breadth of properties required from bone graft materials is to provide multiple bone graft materials with the intention to apply each to a specific class of indications. If the clinician requires a mixture of properties or attributes, the clinician must mix the currently available products from different manufacturers to obtain a desirable set of attributes or move on to another product already designed with the right set of attributes. Thus, in the present invention, a system of products that may be used either independently or mixed with any of the other constituents in the system is provided. A list of the constituents envisioned include: a bone material component available as a shaped particle, a bioceramic component with osteoconductive properties that is available as a shaped particle; suspension material that aids primarily in the delivery of the shaped particles; a compliant shaped particle with improved mechanical properties that mimics the compliance of allograft cancellous bone; a fibrin matrix that can act as a carrier as with the suspension material but can provide some enhancement to bone healing, as well as act as a carrier for the following items; antibiotics, cancer therapy, osteoporosis therapies, or therapies for other bone mineralization disorders that can affect the overall efficacy of a bone graft material depending on the complications associated with the graft procedure; growth factors, bone morphogentic proteins, or protein fragments that can further enhance bone healing and/or have a specific high affinity for the fibrin matrix (these factors may utilize a wide variety of pathways to meet the end results such as influencing the development of mesenchymal stem cells, growth and reproduction of osteoblast/osteoclast/osteocytes, chemotoxic agents that encourage mitogenesis and re-population by the osteoblasts/osteoclasts/osteocytes, angiogenic agents, etc.); cells which may also be delivered using a fibrin matrix which are beneficial to bone healing such as osteoblasts, osteoclasts, and/or osteocytes; allograft bone and bone products; and other biological agents.

In a preferred embodiment these components are compatible with autograft. It is generally known that clinicians prefer to use autograft over existing synthetics since it is the tissue that is trying to be emulated. Clinicians will mix in autograft and/or blood to fill in the missing aspects or properties (primarily to capture the bioactive aspects) of the currently available products in an object of the present invention.

The present bone graft system invention offers several improvements over current bone graft substitutes: all components may be resorbable/degradable in vivo (current products offered include both resorbable/degradable and permanent structure); interlocking structure increases mechanical strength and stability of the granular structure (particularly under shear forces) relative to the current designs of random and regular, non-interlocking structures; interlocking structure that also maintains open, interconnected porosity which allows the individual shaped particles to be dense and therefore less likely to chip and break than current porous (ceramic) structures which are friable and weak; dense shaped particles will not adhere to soft tissues as will the currently available porous ceramic structures; offering product as a shaped particle allows the clinician to fill a large range of defect sizes, whereas current products offer granule and block forms; a multi-component system allows the clinician to tailor the bone graft to the needs of the patient without having to utilize many different product offerings (current products do not offer this flexible, systematic approach); the addition of antibiotics to the system allows the clinician to graft at an earlier stage in cases where infection is a concern; and the addition of biological factors which may hasten the bone healing process to or onto a component of the system of the invention can provide superior mechanical support which will offer an advantage over the current delivery system (a collagen sponge) for such molecules.

The integral advantage of a system of the invention is that it eliminates the need to develop a specific product for each specific indication. The clinician can now mix/match the components of the system as needed to provide the desirable mixture of attributes, thus having the ability to tailor or design a bone graft product for each patient to suit his or her unique needs and specific complications. This results in a lower cost to the patient who will be charged only for the products used.

Flexibility in pharmaceutical choice to match infectious agents is also an advantage of the present invention. In the case of antibiotics, the clinician can choose the appropriate antibiotic based on the culture results from the wound. In the case of some currently available products, the clinician has only one choice for an antibiotic (tobramycin).

There is also provided greater ease of storage and lower distribution costs as compared to products which directly incorporate bioactive proteins, cells, or pharmaceuticals. These ‘active’ ingredients have specific storage conditions and limited shelf lives. If the products are pre-mixed, the manufacturer runs the risk of having to dispose of the entire product at expiration rather than the ‘active’ ingredient with the shorter shelf life. This also eliminates issues caused by the potential for interactions between the ‘active’ ingredients and the device during long storage times.

Furthermore, if the bone graft already contains the pharmaceutical or bioactive protein or cells, then the product may be limited in its use to treat larger defects for fear of over dosing. Similar issues are encountered in treating small defects where the dose may be too small to have a beneficial outcome. Giving the clinician the ability to set the dose allows that the proper dose will be used in all cases.

V. Addition of Biological Agents to the System

In a preferred embodiment of the present invention a biological agent is included in the bone material particle, on the bone material particle, or both, and/or in the suspension material. Examples include antibiotics, growth factors, fibrin, bone morphogenetic factors, angiogenic factors, bone growth agents, chemotherapeutics, growth factor accessory or binding proteins, cells, pain killers, bisphosphonates, strontium salt, fluoride salt, magnesium salt, and sodium salt.

In contrast to administering high doses of antibiotic orally to an organism, the present invention allows antibiotics to be included within or on the particle and/or within the suspension material of the composition for a local administration. This reduces the amount of antibiotic required for treatment of or prophalaxis for an infection. Administration of the antibiotic by the suspension material in a composition would also allow less diffusing of the antibiotic, particularly if the antibiotic is contained within a fibrin matrix. Alternatively, the particles of the present invention may be coated with the antibiotic and/or contained within the particle or the suspension material. Examples of antibiotics are tetracycline hydrochloride, vancomycin, cephalosporins, aminoglycocides such as tobramycin and gentamicin, and quinolone antibiotics such as ciprofloxacin.

Growth factors may be included in the suspension material for a local application to encourage bone growth. Examples of growth factors which may be included are platelet derived growth factor (PDGF), transforming growth factor ax (TGF-α), insulin-related growth factor-I (IGF-I), insulin-related growth factor-II (IGF-II), fibroblast growth factor (FGF), beta-2- microglobulin (BDGF II) epidermal growth factor (EGF), keratinocyte growth factor (KGF), and bone morphogenetic protein (BMP). The particles of the present invention may be coated with a growth factor and/or contained within the particle or the suspension material.

Proteins or agents which are accessory to and/or bind to growth factor may be used in the present invention. Examples of the growth factor binding/accessory protein includes follistatin, osteonectin, sog, chordin, dan, cyr61, thrombospondin, type IIa collagen, endoglin, cp12, nell, crim, acid-1 glycoprotein, and alpha-2HS glycoprotein.

In some embodiments, the compositions of the present invention include a cell, such as an osteoblast, endothelial cell, fibroblast, adipocyte, myoblast, mesenchymal stem cell, chondrocyte, multipotent stem cell, pluripotent stem cell and totipotent stem cell, or a musculoskeletal progenitor cell.

Bone morphogenetic factors may include growth factors whose activity is specific to osseous tissue including proteins of demineralized bone, or DBM (demineralized bone matrix), and in particular the proteins called BP (bone protein) or BMP (bone morphogenetic protein), which actually contains a plurality of constituents such as osteonectin, osteocalcin and osteogenin. The factors may coat the shaped particles of the present invention and/or may be contained within the particles or the suspension material.

Angiogenic factors may be included on the particle or in the particle, or both. Some examples of angiogenic factors include monobutyrin, dibutyrin, tributyrin, butyric acid, vascular endothelial growth factor (VEGF), erucimide, synthetic thymosin Beta 4(TB4), synthetic peptide analogs to heparin binding proteins, nicotine, nicotinamide, spermine, angiogenic lipids, ascorbic acid and derivatives thereof and thrombin, including analogs and peptide fragments thereof.

Bone growth agents may be included within the suspension material of the composition of the invention in a specific embodiment. For instance, nucleic acid sequences which encode an amino acid sequence, or an amino acid sequence itself may be included in the suspension material of the present invention wherein the amino acid sequence facilitates bone growth or bone healing. As an example, leptin is known to inhibit bone formation (Ducy et al., 2000). Any nucleic acid or amino acid sequence which negatively impacts leptin, a leptin ortholog, or a leptin receptor may be included in the composition. As a specific example, antisense leptin nucleic acid may be transferred within the composition of the invention to the site of a bone deficiency to inhibit leptin amino acid formation, thereby avoiding any inhibitory effects leptin may have on bone regeneration or growth. Another example is a leptin antagonist or leptin receptor antagonist.

The nucleic acid sequence may be delivered within a nucleic acid vector wherein the vector is contained within a delivery vehicle. An example of such a delivery vehicle is a liposome, a lipid or a cell. In a specific embodiment the nucleic acid is transferred by carrier-assisted lipofection (Subramanian et al., 1999) to facilitate delivery. In this method, a cationic peptide is attached to an M9 amino acid sequence and the cation binds the negatively charged nucleic acid. Then, M9 binds to a nuclear transport protein, such as transportin, and the entire DNA/protein complex can cross a membrane of a cell.

An amino acid sequence may be delivered within a delivery vehicle. An example of such a delivery vehicle is a liposome. Delivery of an amino acid sequence may utilize a protein transduction domain, an example being the HIV virus TAT protein (Schwarze et al., 1999).

In a preferred embodiment the biological agent of the present invention has high affinity for a fibrin matrix.

In a specific embodiment, the particle of the present invention may contain within it or on it a biological agent which would either elute from the particle as it degrades or through diffusion.

The biological agent may be a pain killer. Examples of such a pain killer are lidocaine hydrochloride, bipivacaine hydrochloride, and non-steroidal anti-inflammatory drugs such as ketorolac tromethamine.

Other biological agents which may be included in the suspension material or contained on or in the particles of the present invention are chemotherapeutics such as cis-platinum, ifosfamide, methotrexate and doxorubicin hydrochloride. A skilled artisan is aware which chemotherapeutics would be suitable for a bone malignancy.

Another biological agent which may be included in the suspension material or contained on or in the particles of the present invention is a bisphosphonate. Examples of bisphosphonates are alendronate, clodronate, etidronate, ibandronate, (3-amino-1-hydroxypropylidene)-1,1-bisphosphonate (APD), dichloromethylene bisphosphonate, aminobisphosphonatezolendronate and pamidronate.

The biological agent may be either in purified form, partially purified form, commercially available or in a preferred embodiment are recombinant in form. It is preferred to have the agent free of impurities or contaminants.

VI. Addition of Fibrinogen to the Composition

It is advantageous to include into the composition of shaped particles and suspension material any factor or agent that attracts, enhances, or augments bone growth. In a specific embodiment the composition further includes fibrinogen, which, upon cleaving by thrombin, gives fibrin. In a more preferred embodiment Factor XIII is also included to crosslink fibrin, giving it more structural integrity.

Fibrin is known in the art to cause angiogenesis (growth of blood vessels) and in an embodiment of the present invention acts as an instigator of bone growth. It is preferred to mimic signals which are normally present upon, for instance, breaking of bone to encourage regrowth. It is known that fibrin tends to bind growth factors which facilitate this regrowth.

In an object of the present invention the inclusion of fibrin into the composition is twofold: 1) to encourage bone growth; and 2) to act as a delivery vehicle.

The fibrin matrix is produced by reacting three clotting factors—fibrinogen, thrombin, and Factor XIII. These proteins may be manufactured using recombinant techniques to avoid issues associated with pooled-blood products and autologous products. Currently, the proteins are supplied in a frozen state ready for mixing upon thawing. However, lypholization process development allows that the final product will either be refrigerated or stored at room temperature and reconstituted immediately prior to use. In a preferred embodiment the clotting factors are recombinant in form.

Only fibrinogen and thrombin are required to produce a fibrin matrix in its simplest form. However, the addition of Factor XIII provides the ability to strengthen the matrix by means of cross linking the fibrin fibrils. Specific mixtures of the three proteins may be provided to generate the appropriate reaction time, degradation rate, and elution rate for the biological agents.

Modifications can be made by altering the fibrin component. One expected modification would be to use hyaluronic acid or a collagen gel instead of or in addition to a fibrin component. Other variations may be inclusion of additional clotting factors in the fibrin matrix. Additional examples of clotting factors are known in the art and may be used, but in a specific embodiment they are clotting factors relevant to a bone disorder. The clotting factors may be purified, partially purified, commercially available, or in recombinant form. In a specific embodiment thrombin alone is used with the patient's own blood or bone marrow aspirate to produce a fibrin matrix.

In a specific embodiment a biological agent as described above is contained within the fibrin matrix.

VII. Manufacturing of the Compositions

It is an object of the present invention to provide apparatus and methods to manufacture a bone graft substitute through powder compaction of a bone material powder into a shape. Although the bone material powder may be an allograft material, a synthetic material, a ceramic material, a polymer material, or a combination thereof, it is preferably demineralized bone matrix. A preferred shape is a jack, such as a JAX® particle.

The method of manufacturing the BGS could be done by standard molding or injecting techniques, although preferably it includes processing such as pulverizing, compressing, compacting, pressably contacting, packing, squeezing, tamping, or squashing a bone material powder into the desired shape. The method preferably utilizes powder compaction, which a skilled artisan recognizes is a process well known in metal and ceramic powder processing. A processing aid composition is preferably utilized to facilitate compaction of the material and release of the product from the die. A releasing agent may also be used to release the composition. The releasing agent, such as stearic acid, may be coated or painted onto the die or could be in the particle, or both.

In one embodiment of the present invention, the method includes obtaining a bone material, such as from a donor, cadaver, and the like, processing the material to produce a bone material powder, which a skilled artisan recognizes is preferably to a consistency which is conducive to compaction and generation of a product which is substantially non-friable. The particles are preferably substantially homogeneous in size. The powder is then subjected to a powder compaction process.

The powder compaction process preferably utilizes a withdrawal press. The withdrawal press may comprise a lower punch assembly, an upper punch assembly, and a moveable die. A skilled artisan also recognizes the press will comprise other parts standard in the art, such as a means to fill a die cavity with the powder, and so on. The lower punch assembly may comprise at least one of a stationary punch and a moveable punch; a skilled artisan recognizes this is referred to as a “dual punch”. The moveable punch preferably is vertically moveable about the stationary punch. Similarly, an upper punch assembly may comprise at least one of a stationary punch and a moveable punch, wherein the moveable punch preferably is vertically moveable about the stationary punch. In a preferred embodiment, the apparatus comprises a dual lower punch and an upper punch. In some embodiments, the upper is a single punch but is moving up and down in coordination with the lower punch(es); one of the lower punches of the “dual punch” is stationary. That is, if there is only one lower punch, this one is stationary.

The die is preferably moveable, although it may be stationary, and is generally located, during processing, between the lower and upper punch assemblies. It is preferably in alignment with at least one of a lower and upper punch. The die preferably has at least one cavity, and also preferably is shaped corresponding to the desired generated shape of the particle and to permit the corresponding punches to fit in the cavity.

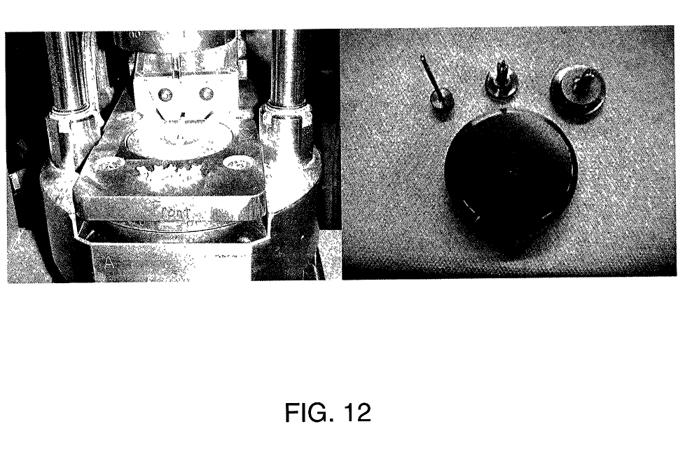

The surfaces of the punches which contact the powder material are preferably configured with a contour or shape that imparts the desired shape onto the powder upon contact with the material. The shape may be a jack, a tablet, a strip, a block, a cube, a pellet, a pill, a lozenge, a sphere, or a ring. The shape of the punches may be that which will impart a jack shape, such as is demonstrated in FIG. 12. The shape is preferably a jack such as a JAX® particle. In one embodiment of the present invention, one of the punches may impart a jack shape and the other punch may have a generally flat surface, although the resulting product will still result in a jack shape.

In the process, the moveable die and punch assemblies are provided. The powder is introduced into a cavity in the die and the die is positioned generally in alignment with at least one of the punches. In a preferred embodiment, the die is positioned generally above the stationary lower punch. In a specific embodiment, a moveable upper punch pressably contacts the powder in opposition to the moveable lower punch and stationary lower punch. A moveable lower punch moves to pressably contact the powder in opposition to an upper punch. In a specific embodiment, the moving steps occur generally simultaneously, and in other specific embodiments, the moving steps occur in sequence. The steps of moving the upper and lower punches preferably effect a substantially uniform distribution of pressure within the powder. The uniformity of the pressure distribution across the surface of the powder is desirable because it is the best way to ensure the resulting product is structurally sound. The moving steps thus form the powder into the desired shaped BGS.

The moving steps preferably apply a force in the range of about 0.2 to about 5 tons, more preferably about 0.2 to about 2 tons, and most preferably about 0.5 to about 1 ton. The force may be greater, and a skilled artisan recognizes that the upper limit is determined by the critical density of the powder.

In one embodiment of the present invention, there are an apparatus and method for manufacturing a bone graft substitute wherein a stationary lower punch has a top surface, a moveable lower punch vertically moveable about the stationary lower punch has a top surface, and a moveable upper punch, such that when the moveable upper punch moves in opposition to the moveable lower punch to pressably contact the powder in the die cavity the top surface height of the moveable lower punch is above the top surface height of the stationary lower punch.

In one embodiment of the present invention, there is a method for manufacturing a bone graft substitute wherein the steps comprise providing a first punch assembly having a first contact surface configured to effect a relief profile onto a first surface of the bone material powder, preferably a demineralized bone matrix, a second punch assembly having a second contact surface, and a moveable die having at least one cavity; introducing the powder into the cavity; positioning the moveable die generally in alignment with the first punch assembly; moving at least a portion of the first punch assembly to pressably contact the powder in opposition to the second punch assembly to effect the desired relief profile on the first surface thereof; and moving at least a portion of the second punch assembly to pressably contact the powder in opposition to the first punch assembly, whereby the moving steps form the powder into the shaped bone graft substitute.

The contact surface area of the first punch assembly is generally equivalent to a contact surface area of the second punch assembly such that the moving steps apply a substantially uniform pressure distribution to the powder. In a specific embodiment, the first punch assembly includes a stationary punch and a moveable punch, such that the steps of moving the first punch assembly includes moving the moveable punch to pressably contact the powder. In another specific embodiment, the second punch assembly includes a stationary punch and a moveable punch, such that the steps of moving the second punch assembly includes moving the moveable punch to pressably contact the powder.

In another embodiment of the present invention, there is an apparatus for manufacturing a bone graft substitute from a bone material powder wherein the apparatus comprises a first punch assembly having a first contact surface having a profile configured to effect a relief profile onto a surface of the bone material powder; a second punch assembly having a second contact surface, the second contact surface positioned in general alignment with the first contact surface; and a moveable die having at least one cavity, the moveable die being positionable generally in between the first and second punch assemblies.

### EXAMPLE 1

### Testing of Shaped Particles

The assessment of the shaped particles was based on two tests designed to address interlocking of the particles and application to a clinical-type case.

‘Slump’ test—measure the ability of a pile of bone graft granules to maintain its height before and after vibration.

Push-thru test—measure the resistance to push-thru of an agglomeration of bone graft granules through a cylindrical defect in a porous foam block, which is a lab model used for human cancellous bone.

The goal was to determine which of the designs provided the most interlocking that was also an improvement over a design comparable to a commercially available tablet-shaped product.

Equipment:

| | |
|---|---|
| A) ‘Slump’ test | B) Push-Thru test |
| Tablets, 28 mL | Tablets, 50 mL |
| Shaped particle designs, 28 mL | Shaped particle designs, 50 mL of |
| of each 100 mL graduated cylinder | each Tinius-Olsen screw driven |
| (EXAX, No. 20025) | mechanical test frame and |
| Scale (Mettler Toledo, AT261) | #2000 recorder |
| Vibrating, electronic pencil (Ideal | Porous foam block (General Plastics |
| Industries, Electric Marker) | Manufacturing Company, FR3703) |
| Funnel (half angle 28°) | Polyethylene plunger and stopper |
| Cuplike container (half angle 12°, | Image pro Plus Software (Media |
| base diameter 1.125″) | Cybernetics, V 3.0.1) |
| Ring stand | |
| Height gage (Mitutuyo, No. | |
| 192-112) | |
| Base plate (1 × 6 × 6 inch | |
| cold-rolled steel) | |
| Watch with second hand | |

Three different shaped particles of the present invention (Six-armed shaped particle, flared to bulb at end of arms of X-Y plane (FIG. 8); Five-armed shaped particle, flared to bulb at end of arm s in X-Y place (FIG. 3); Six-armed shaped particle, tapered straight to end of arms in all directions (FIG. 1); and one tablet-shaped geometry similar to commercially available products. The shaped particle designs were manufactured using clay formulation “50-dry”. SLA molds were used to form the design prototypes. The components were all made similarly, though slightly different processing parameters were used with each to insure proper drying and mold release. In the following example, the particles are made from gypsum. However, a skilled artisan recognizes that similar shapes can be made from a bone material.

1. Stereo lithographic models (SLA) were made of molds for each of the three designs.

2. SLA molds were washed and dried.

3. Lubricant was applied to the surface of the SLA molds. Excess was removed with a clean cloth and compressed air.

A. Two lubricants from Slide Products Inc. (Wheeling, Ill.) were used: 42612N, 44712G

B. Pam® (International Home Foods, Parsippany, N.J.) was used as another lubricant

4. Clay formula 50-dry (81.6% gypsum, 1.1% carboxymethyl cellulose, 4.1% glycerin, 13% water) was rolled into sheets (about 1 mm thick), big enough to cover the cavities in the molds.

Gypsum: FG-200, from BPB, Newarks, United Kingdom

carboxymethyl cellulose: 7HF, from Hercules, Wilmington, Del.

Glycerine, USP: GX-195-1, from EM Science, Gibbstown, N.J.

5. The mold halves were closed together and compacted using about 4000 lbs. of force.

6. The molds were heated in a microwave oven to dry the water from the parts.

A. Six-armed shaped particle, flared to bulb at the ends of arms in X-Y plane heated for 4 min. at about 30% power.

B. Five-armed shaped particle X, flared to bulb at the ends of arms in X-Y plane heated for 4:25 min. at about 30% power.

C. Six-armed shaped particle, straight, tapered arms, heated for 3:50 min. at about 30% power.

7. The molds were allowed to cool for approximately one minute.

8. The parts were removed from the mold and trimmed of any flashing using an Exacto knife.

9. The parts were dried in a vacuum dessiccator for several hours prior to further testing.

Slump Test

The slump test was conducted first since it was non-destructive. Equal volumes (28 mL) of each shaped particle design and the tablet samples were measured using a 100 mL graduated cylinder. These equal volumes were weighed to determine the mass of material present.

The test begins by pouring the entire volume of individual shaped particle designs into a starting container. Either a funnel (half angle 28°) or a cuplike container (half angle 12° with a 1.125 inch flat base) was used to contain the shaped bone graft particles and provide a starting shape for the pile. The container was then inverted and placed on a base through which a vibration was applied for five seconds using an electronic, vibrating pencil. The vibration was used to settle the shaped bone graft particles into the container of choice and pre-pack them to that shape. Following the vibration, the container was carefully removed. A height gage was used to measure the initial height of the pile. Vibration was then applied to the base plate, causing the pile to settle further. The height gage was used again to measure this new height. The highest particle/tablet was used as the height in all cases. This test was repeated ten times for each design using each of the two containers (funnel and cuplike container). From the data a difference in heights and the percentage change in heights (relative to the initial height of the pile) were calculated.

Table 1 shows the mass data collected for the three shaped particle designs and the tablet geometry. The mass shown is for 28 mL of particles, as measured in a 100 mL graduated cylinder. One data point was collected for each design.

Mass and mass per volume are important and related to the dissolution time and the porosity of the agglomerated granules. If all parameters were equal (material, density, surface-area-to-volume ratios, etc.) it would be expected that the more mass per volume, the lower would be the porosity of the agglomerate and the longer duration it would have before dissolution. The dissolution rate would determine how much material would disappear per unit of time and may also be influenced by the surface-area-to-volume ratio and the material.

TABLE 1
Mass per 28 mL of particles

| Sample | Mass per 28 mL of granules |
|---|---|
| A) Six-armed shaped particle, flared to | 17.2175 |
| bulb at end of arms of X-Y plane | |
| B) Five-armed shaped particle, flared to | 20.2567 |
| bulb at end of arms in X-Y place | |
| C) Six-armed shaped particle, tapered | 21.2140 |
| straight to end of arms in all directions | |
| D) Tablet geometry | 31.3437 |

Table 2 shows the summarized results for the slump tests performed on each of the different sample geometries using the funnel for a starting form. Each sample was measured ten times. It was proposed that maximizing the starting height and the height after vibration and minimizing the change in height and percent change in height were the ideal cases. The best value for the shaped particle designs tested for each parameter is in bold. The tablets did not form a pile (tablets fell to only one or two layers high) when the supporting container was removed, qualitatively indicating poor interlocking relative to other samples.

TABLE 2
Summarized results for the slump tests using
the funnel for starting form.

| | | | | Percent |
|---|---|---|---|---|
| | | | | change in |
| | | | | height, |
| | | | | relative to |
| | Starting | Height after | Change in | starting |
| | height, H1 | vibration, H2 | height, δ | height |
| Sample | (inches) | (inches) | (inches) | (inches) |
| A) Six-armed shaped | 1.275 ± | 0.821 ± | 0.454 ± | 35.138 ± |
| particle, flared to bulb | 0.109 | 0.070 | 0.147 | 8.072 |
| at end of arms of X-Y | | | | |
| plane (n = 10) | | | | |
| B) Five-armed shaped | 1.223 ± | 0.806 ± | 0.418 ± | 33.350 ± |
| particle, flared to bulb | 0.161 | 0.069 | 0.146 | 8.675 |
| at end of arms in X-Y | | | | |
| plane (n = 10) | | | | |
| C) Six-armed shaped | 1.114 ± | 0.829 ± | 0.285 ± | 24.734 ± |
| particle, tapered | 0.158 | 0.054 | 0.128 | 7.955 |
| straight to end of arms | | | | |
| in all directions | | | | |
| (n = 10) | | | | |
| D) Tablet geometry, | 0.662 ± | 0.578 ± | 0.084 ± | 12.342 ± |
| (n = 10) | 0.055 | 0.032 | 0.056 | 6.981 |

Funnel

6-arm/bulb arm:

| | H1 (inches) | H2 (inches) | Δ |
|---|---|---|---|
| T1 | 1.22 | 0.885 | 0.335 |
| T2 | 1.56 | 0.738 | 0.822 |
| T3 | 1.28 | 0.81 | 0.470 |
| T4 | 1.18 | 0.76 | 0.420 |
| T5 | 1.18 | 0.75 | 0.430 |
| T6 | 1.3 | 0.790 | 0.51 |
| T7 | 1.283 | 0.80 | 0.483 |
| T8 | 1.121 | 0.926 | 0.195 |
| T9 | 1.255 | 0.823 | 0.432 |
| T10 | 1.285 | 0.929 | 0.356 |

5- arm:

| | H1 (inches) | H2 (inches) | Δ |
|---|---|---|---|
| T1 | 1.344 | 0.093 | 0.441 |
| T2 | 1.185 | 0.830 | 0.355 |
| T3 | 1.180 | 0.75 | 0.430 |
| T4 | 1.150 | 0.801 | 0.349 |
| T5 | 1.760 | 0.89 | 0.470 |
| T6 | 1.39 | 0.787 | 0.603 |
| T7 | 1.103 | 0.656 | 0.447 |
| T8 | 1.472 | 0.823 | 0.649 |
| T9 | 0.959 | 0.812 | 0.147 |
| T10 | 1.090 | 0.806 | 0.284 |

6 arm/straight arm:

| | H1 | H2 | Δ |
|---|---|---|---|
| T1 | 1.132 | 0.890 | 0.242 |
| T2 | 1.269 | 0.862 | 0.407 |
| T3 | 1.219 | 0.801 | 0.418 |
| T4 | 0.93 | 0.786 | 0.144 |
| T5 | 0.967 | 0.849 | 0.118 |
| T6 | 1.049 | 0.791 | 0.258 |
| T7 | 1.050 | 0.789 | 0.261 |
| T8 | 1.451 | 0.93 | 0.521 |
| T9 | 1.020 | 0.829 | 0.191 |
| T10 | 1.053 | 0.760 | 0.293 |

Tablet:

| | H1 | H2 | Δ |
|---|---|---|---|
| 1 | 0.634 | 0.576 | 0.058 |
| 2 | 0.670 | 0.641 | 0.029 |
| 3 | 0.681 | 0.543 | 0.138 |
| 4 | 0.618 | 0.540 | 0.078 |
| 5 | 0.637 | 0.559 | 0.078 |
| 6 | 0.690 | 0.574 | 0.116 |
| 7 | 0.644 | 0.594 | 0.005 |
| 8 | 0.613 | 0.551 | 0.062 |
| 9 | 0.799 | 0.591 | 0.208 |
| 10 | 0.635 | 0.609 | 0.026 |

Table 3 shows the summarized results for the slump tests performed using the cuplike container for a starting form. As with the slump test using the funnel for a starting containing, maximizing the start height and the height after vibration and minimizing the change in height and percent change in height were the ideal cases. The best value for the shaped particle designs tested in each column in bold.

TABLE 3
Cuplike Container
Summarized results for the slump tests using the cuplike
container for starting form.

| | | | | Percent |
|---|---|---|---|---|
| | | | | change in |
| | | | | height, |
| | | | | relative to |
| | Starting | Height after | Change in | starting |
| | height | vibration | height | height |
| Sample | (inches) | (inches) | (inches) | (inches) |
| A) Six-armed shaped | 0.970 ± | 0.860 ± | 0.111 ± | 11.184 ± |
| particle, flared to bulb | 0.056 | 0.027 | 0.051 | 4.696 |
| at end of arms of X-Y | | | | |
| plane | | | | |
| (n = 10) | | | | |
| B) Five-armed shaped | 0.997 ± | 0.844 ± | 0.1530.063 | 15.194 ± |
| particle, flared to bulb | 0.051 | 0.056 | | 5.894 |
| at end of arms in X-Y | | | | |
| plane | | | | |
| (n = 10) | | | | |
| C) Six-armed shaped | 0.907 ± | 0.744 ± | 0.133 ± | 14.435 ± |
| particle, tapered | 0.062 | 0.052 | 0.067 | 6.854 |
| straight to end of arms | | | | |
| in all directions | | | | |
| (n = 10) | | | | |
| D) Tablet geometry, | 0.516 ± | 0.441 ± | 0.075 ± | 14.361 ± |
| (n = 10) | 0.049 | 0.040 | 0.030 | 5.077 |

Actual Test Data are as Follows:

6 Arm/Bulb Arm

| | H1 | H2 | Δ |
|---|---|---|---|
| 1 | 1.070 | .870 | 0.20 |
| 2 | 0.975 | .826 | 0.149 |
| 3 | 1.005 | .880 | 0.125 |
| 4 | 0.891 | .849 | 0.042 |
| 5 | 0.905 | .821 | 0.084 |
| 6 | 0.951 | .875 | 0.076 |
| 7 | 0.949 | .886 | 0.063 |
| 8 | 0.940 | .875 | 0.065 |
| 9 | 1.038 | .890 | 0.148 |
| 10 | 0.979 | .826 | 0.153 |

5-arm:

| | H1 | H2 | Δ |
|---|---|---|---|
| 1 | 1.005 | 0.798 | 0.207 |
| 2 | 0.935 | 0.815 | 0.055 |
| 3 | 0.934 | 0.880 | 0.054 |
| 4 | 1.032 | 0.823 | 0.209 |
| 5 | 1.020 | 0.894 | 0.126 |
| 6 | 0.994 | 0.804 | 0.190 |
| 7 | 1.062 | 0.856 | 0.206 |
| 8 | 1.030 | 0.802 | 0.228 |
| 9 | 0.915 | 0.801 | 0.114 |
| 10 | 1.041 | 0.968 | 0.073 |

tablet:

| | H1 | H2 | Δ |
|---|---|---|---|
| 1 | 0.466 | 0.411 | 0.055 |
| 2 | 0.469 | 0.419 | 0.05 |
| 3 | 0.560 | 0.471 | 0.089 |
| 4 | 0.590 | 0.472 | 0.118 |
| 5 | 0.511 | 0.470 | 0.041 |
| 6 | 0.540 | 0.40 | 0.14 |
| 7 | 0.467 | 0.412 | 0.055 |
| 8 | 0.457 | 0.379 | 0.078 |
| 9 | 0.540 | 0.406 | 0.134 |
| 10 | 0.562 | 0.492 | 0.070 |

Data from the two slump tests indicated that for the test using the funnel for support and shape of the initial pile, the six-armed shaped particle with simple tapers was seen to be better than the other designs. In the test using the cuplike container, the six-armed shaped particle with the arms in the X-Y plane flared to bulbs was seen as the better design.

Push-thru Test

The push-thru test was a mechanical test performed using a Tinius-Olsen (Willow Grove, Pa.) screw-driven mechanical test frame. Once tested using this procedure, the sample parts and the defects in the porous blocks were considered to be damaged and not valid for additional testing.

A polyethylene stopper was placed into the bottom of the pre-drilled, 0.750″ hole (thru) in the porous foam block. Then, a volume (approximately 8 mL) of shaped particle is added to the hole and the top plunger is inserted. The correct amount of shaped particles are added when the plunger sits such that the fill mark just shows above the level of the top of the porous foam block. The test block with plunger, stopper and shaped particles are then transferred to the test frame. The part to be tested is situated such that the stopper is over a solid block to temporarily block the shaped particle and stopper from falling through. A pre-load of ten pounds of force is then applied at a rate of 0.1 inches/minute. The pre-load is then removed and the stopper is positioned over an opening such that the plunger can press against the shaped particles and the majority of resistance comes from frictional forces between the shaped particle and the shaped particle and the walls. Additional resistance is expected between the stopper/plunger and the walls, but this should be small and consistent in all tests performed. Load is reapplied at a rate of 0.1 inches/minute until the resisting load drops to zero and the granules are gone from the test block. Data is recorded using a load/displacement graph. This test was repeated five times for each of the three shaped particle designs and three times for the tablet geometry.

The data was analyzed using Image Pro Plus software (Media Cybernetics) to determine the area under the curves. The assumption was made that the load and displacement axes were both to the same scale (displacement) which means that the value calculated for area under the curve is not truly energy. The values of the area under the load-displacement curve are useful for comparing one against the other and to show relatively which design required more energy to force the granules through the block.

Table 4 shows the summarized results for the push-thru testing on each of the different geometries.

TABLE 4
Summarized results for the push-thru tests.

| | Area under load | |
|---|---|---|
| | vs. | Percentage vs. |
| | displacement | six-armed, |
| Sample | (in²)** | tapered |
| A) Six-armed shaped particle, | 0.057 ± 0.015 | 0.655 |
| flared to bulb at end of arms of | | |
| X-Y plane (n = 5) | | |
| B) Five-armed shaped particle, | 0.058 ± 0.009 | 0.667 |
| flared to bulb at end of arms in X-Y | | |
| plane (n = 5) | | |
| C) Six-armed shaped particle, tapered | 0.087 ± 0.019 | 1.000 |
| to end of arms in all directions | | |
| (n = 5) | | |
| D) Tablet geometry, “OsteoSet ®- | 0.003 ± 0.003 | 0.034 |
| like” shape (n = 10) | | |

**area under curve was measured using the Image Pro software package, with both axes (load and displacement) calibrated as inches. This is not a true energy measurement, but serves for comparative purposes.

**area under curve was measured using the Image Pro software package, with both axes (load and displacement) calibrated as inches. This is not a true energy measurement, but serves for comparative purposes.

Maximizing the area under the load/displacement curve was ideal—indicating the most energy required to overcome resistance of interlocking and friction. The maximum value was found with the six-armed shaped particle that was tapered on all arms and is listed in bold in the table. Difference in the push-thru resistance between this design and each of the other three designs was found to be statistically significant (student t-test, two tail, unequal variance, p<0.05).

Observations during the testing showed that all three shaped particle designs resisted push-thru similarly—the granules interlocked with themselves and the walls of the foam block to resist the motion of the plunger through nearly the entire thickness of the test block. The tablet geometry did not offer much resistance, with only a short travel distance required before all of the granules fell out of the bottom of the test block.

The tested granules can be listed in order of decreasing mass per 28 mL volume: tablet geometry, six-armed shaped particle with tapered arms, five-armed shaped particle, and six-armed shaped particle flared to bulb at the end of arms in X-Y plane.

The conclusions of the slump testing and push-thru testing are as follows:

Slump testing of the different designs indicated that the test using the funnel (28° half angle) showed the six-armed shaped particle with tapered arms to be the best. The test using the cuplike container (12° half angle, 1.125″ base) showed the six-armed shaped particle with X-Y plane arms flared to bulbs to be the best. It was also seen that the tablets behaved qualitatively worse compared to any of the shaped particle designs, failing to interlock and retain much of the original pile height.

Push-thru testing showed that the six-armed shaped particle with tapered arms offered the most resistance to push the granules all the way through the porous foam test block. The other shaped particle designs both required about ⅓ less energy to push the granules through the same block. The tablets required only about 3% of the energy required to push-thru the six-armed shaped particle with tapered arms. All of the shaped particle designs were observed to resist push-thru until the plunger was nearly all of the way through the test block. The tablet geometries fell through after the plunger traveled only a short distance through the block.

### EXAMPLE 2

### Powder Compaction of Demineralized Bone Matrix

A skilled artisan recognizes that there are multiple means in the art to manufacture shaped particles, such as JAX®, of a bone material. Examples include casting/injection molding between molds, pressing, tableting, compressing, compacting, pressably contacting, packing, squeezing, tamping, or squashing a bone material powder into the desired shape. A skilled artisan also recognizes that with casting/injection molding techniques, a slurry would be required for the manufacturing, and the moisture may impart deleterious effects onto the DBM. Thus, the present invention is an improvement over other known methods in the art.

Human DBM (HDBM) in powder/chips form was obtained from a bone tissue bank, mechanically ground, and sieved through a #60 mesh (<250 μm particle size). Two different batches were processed. Each ground and sieved HDBM was then blended with 2% (in weight) stearic acid, the latter being used as processing aid in the powder compaction process:

| | HDBM (98%) | | Stearic Acid (2%) | |
|---|---|---|---|---|
| ALLOJAX100-a | 7.6582 | g | 0.1562 | g |
| ALLOJAX100-b | 19.6 | g | 0.4 | g |

A powder compaction press (withdrawal type) was used to compress the blends. Special tooling had been made to allow uniform distribution of compressive forces during the compaction process. This involved a one-piece upper punch, two lower punches, and a floating die (FIG. 12). A compression force between 0.6 and 0.7 tons was used.

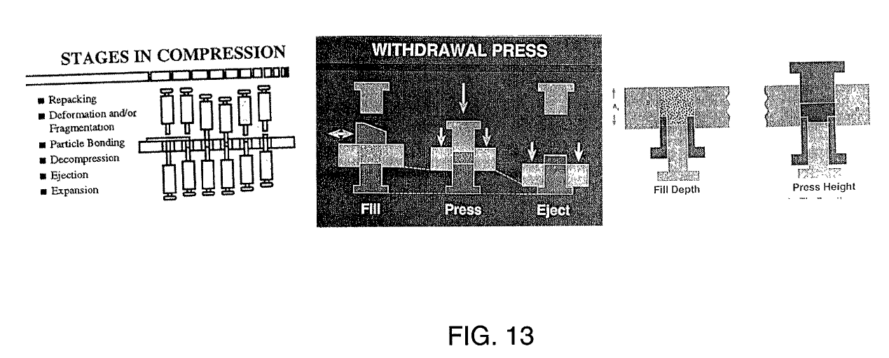

The powder compaction process is unique to produce bone graft substitutes and bone void fillers. Previous BGS products have been produced using a tableting process. Tablet processing consists of a simple pressing action with a lower punch pressing the powder blend against a stationary, or sometimes translating, upper punch through a stationary die. Tableting typically utilize a tableting press. For more complicated shapes, tableting does not allow for a uniform distribution of pressures within the granules and therefore does not allow for the production of intricate shapes, such as a six-arm JAX® granule. Powder compaction is an advanced manufacturing process that allows for a uniform distribution of pressures during compaction, therefore allowing for the production of intricate shapes. In addition, specific tooling is required that allows several relative translations between several punches to distribute the compaction pressures. In powder compaction, the upper punch, lower outer punch and die are translating; the lower inner punch is stationary but because of the relative motion of the punches and die, the pressure is evenly distributed within the powder compacted part. Powder compaction requires the use of a withdrawal press. A schematic comparing the tableting to the powder compaction process is shown in FIG. 13.

FIG. 13 illustrates the differences between (a) conventional tableting and (b, c) the powder compaction used in the novel application to make bone graft substitutes. In (a), the die is stationary, the top and bottom punches are translating; in (b), a withdrawal press is illustrated, in which the lower punch is stationary, the die and upper punch are translating; in (c), an additional lower outer punch allows for a uniform density distribution for an intricate shape, such as JAX®. Thus, a skilled artisan recognizes that a dual lower punch is useful in the present invention. In alternative embodiments, a dual upper punch is utilized wherein the upper punch is composed of an inner punch and an outer punch.

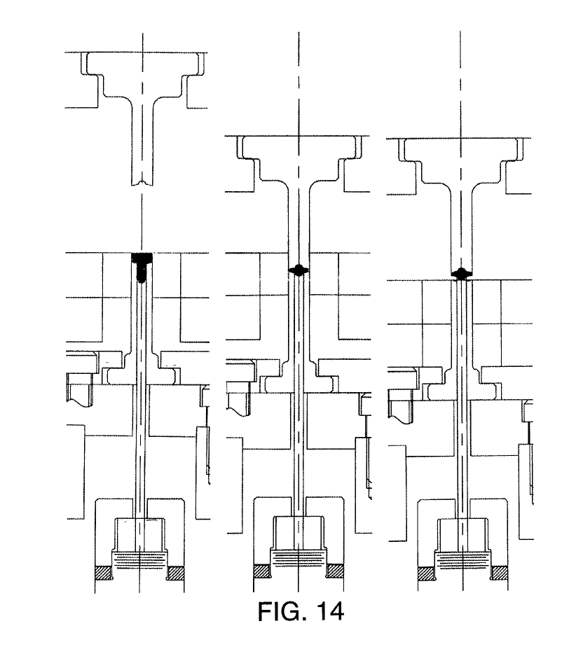

FIG. 14 illustrates a specific embodiment of the present invention, wherein a jack shape is produced through powder compaction. In (a), a die cavity is filled, followed by pressably contacting/compacting the material (b) and ejection of the product (c).

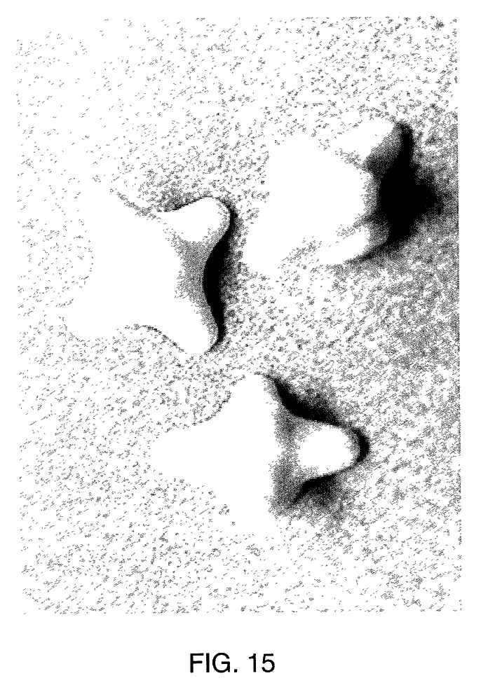

Powder compaction was used to shape DBM powder into an intricate shape (six-arm, JAX®). ALLOJAX100-a compressed poorly; ALLOJAX100-b compressed well and produced a JAX® product that was not friable between fingers (FIG. 15).

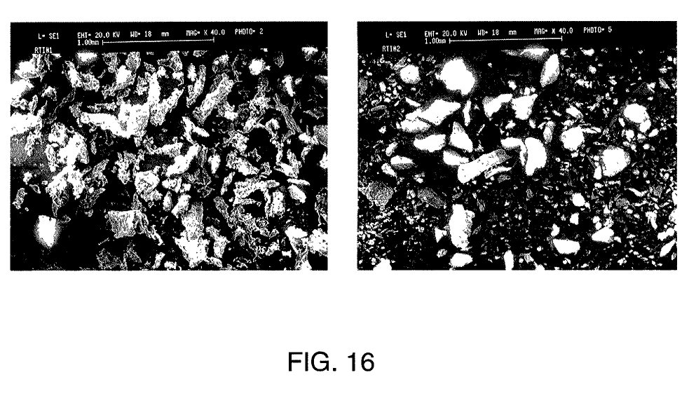

Examination of the two blends and two types of HDBM revealed that batch #-a was composed of mostly acicular, elongated particles, probably mainly cancellous bone tissue, while batch #-b was composed of mostly granules and some fines, probably mainly cortical bone tissue (FIG. 16). The morphology of batch #-b is recommended for powder compaction. Density measurements confirmed the difference between the batches: batch #-b was denser (2.0684 g/cm³) than batch #-a (1.3372 g/cm³).

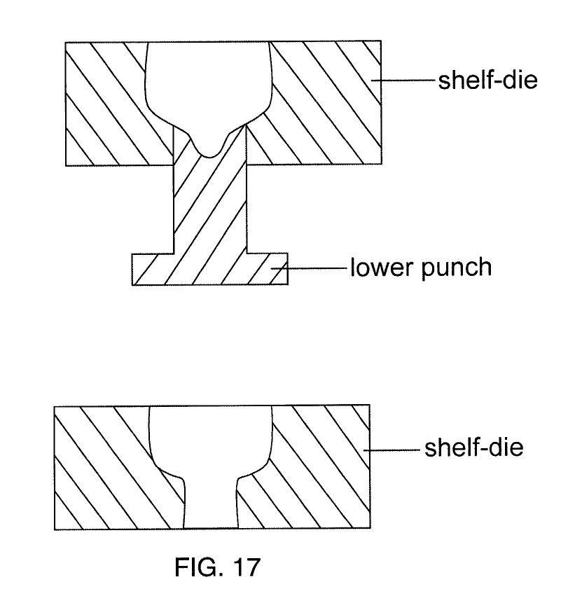

In the previous configuration, the die cavity is a straight cylinder with the walls of this cylinder defining the outer shape of the powder compacted part. In another configuration, the die may be made such that a partial shape of the part to be produced is made into the die. A skilled artisan recognizes this is referred to as a “shelf die”. The lower punch is preferably a single punch whose face matches the shelf die to produce one side of the powder compacted part. A shelf die with lower punch is illustrated in FIG. 17.

### EXAMPLE 3

### Alternative Embodiments

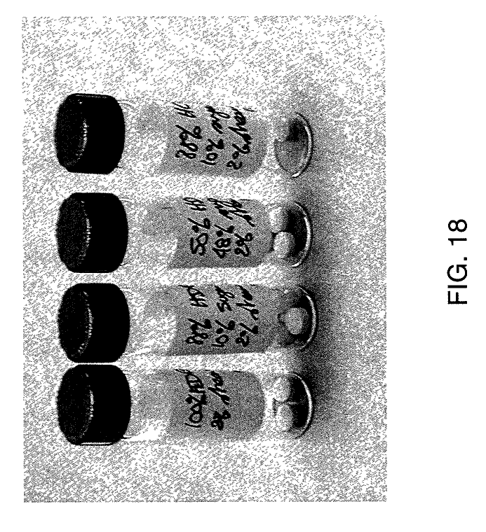

The following blends were successfully compacted into a tablet (about 6 mm in diameter, typical convex shape; FIG. 18):

| Human | Human | | | |
|---|---|---|---|---|
| DBM | Corticocancellous | Calcium Sulfate | Fill Weight | Hardness |
| (%) | Chips (%) | (%) | (mg) | (Kp) |
| 100 | | 0 | 100 | 5.0-6.7 |
| 90 | | 10 | 120 | 6.1-7.2 |
| 50 | | 50 | 120 | — |
| | 90 | 10 | 140 | 2.2-2.4 |
| | 80 | 20 | 140 | 1.6-2.2 |
| | 50 | 50 | 140 | 1.4-1.7 |
| | 90 | 10 | 140 | — |
| | 100 | 0 | 140-170 | — |

For all formulations, the processing aid was stearic acid. The equipment used was a manual hydraulic press, punches used for conventional compression/tableting, and wood blocks for support/guides. Other blends including other allograft (such as human bone or DMB), synthetic or ceramic (such as calcium sulfate or calcium phosphate), or bioactive agents (such as antibiotic, BMPs, acids, and the like), individually or as a mix of two or more of the aforementioned components can potentially be compacted to produce a tablet or a JAX® shape or other shape. A processing aid, or a blend of two or more processing aids (magnesium stearate, calcium stearate, and stearic acid), may be in the compaction process.

### EXAMPLE 4

### Injection Molding

In alternative embodiments of the present invention, the shaped particles are comprised of a ceramic material and manufactured using injection molding techniques.

Injection molding, a skilled artisan recognizes, is used extensively in the plastic industry. Ceramic parts are manufactured with the same injection-molding equipment, but with dies made of harder, more wear-resistant material such as a higher grade tool steel. The feed material generally consists of a mixture of the ceramic powder with a thermoplastic polymer plus a plasticizer, wetting agent, and antifoam agent. The mixture is pre-heated in the barrel of the injection-molding machine to a temperature at which the polymer has a low-enough viscosity to allow flow if pressure is applied. A ram of plunger is pressed against the heated material in the barrel by either a hydraulic, pneumatic, or screw mechanism. The viscous material is forced through an orifice into a narrow passageway that leads to the shaped tool cavity. At the end of the passageway, the strand of viscous material passes through another orifice into the tool cavity. The strand piles on itself until the cavity is full and the material has knit or fused together under the pressure and temperature to produce a homogeneous part. The shaped tool is cooler than the injection-molding mix such that the mix becomes rigid in the tool cavity. The part (“green part”) can be removed from the tool as soon as it is rigid enough to handle without deformation. After injection molding, the plastic and additives are then removed by careful thermal treatments (“brown part”). The ceramic may then be sintered to achieve strength (“final part”).

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

References

All patents and publications mentioned in the specification are indicative of the level of those skilled in the art to which the invention pertains. All patents and publications are herein incorporated by reference to the same extent as if each individual publication was specifically and individually indicated to be incorporated by reference.

Patents

U.S. Pat. No. 4,384,834 issued May 24, 1983.

U.S. Pat. No. 4,619,655 issued Oct. 28, 1986.

U.S. Pat. No. 5,017,122 issued May 21, 1991.

U.S. Pat. No. 5,158,728 issued Oct. 27, 1992.

U.S. Pat. No. 5,366,507 issued Nov. 22, 1994.

U.S. Pat. No. 5,449,481 issued Sep. 12, 1995.

U.S. Pat. No. 5,569,308 issued Oct. 29, 1996.

U.S. Pat. No. 5,603,880 issued Feb. 18, 1997.

U.S. Pat. No. 5,614,206 issued Mar. 25, 1997.

U.S. Pat. No. 5,654,003 issued Aug. 5, 1997.

U.S. Pat. No. 5,762,978 issued Jun. 9, 1998.

U.S. Pat. No. 5,807,567 issued Sep. 15, 1998.

U.S. Pat. No. 6,106,267 issued Aug. 22, 2000.

U.S. Pat. No. 6,030,636 issued Feb. 29, 2001.

U.S. Pat. No. 6,177,125 issued Jan. 23, 2001.

Publications

Ducy, P., Amling, M., Takeda, S., Priemel, M., Schilling, A. F., Beil, F. T., Shen, J., Vinson, C., Rueger, J. M., and Karsenty, G. 2000. Leptin inhibits bone formation through a hypothalamic relay: a central control of bone mass. Cell 100:197-207.

Medica Data International, Inc., Report #RP-591149, Chapter 3: Applications for Bone Replacement Biomaterials and Biological Bone Growth Factors (2000).

Orthopaedic Network News, Vol. 11, No 4, October 2000, pp. 8-10.

Schwarze, S. R., Ho, A., Vocero-Akbani, A. and S. F. Dowdy, 1999. In vivo protein transduction: delivery of a biologically active protein into the mouse. Science 285: 1569-1572.

Subramanian, A., Ranganathan, P. and S. L. Diamond, 1999. Nuclear targeting peptide scaffolds for lipofection of nondividing mammalian cells. Nature Biotechnology 17: 873-877.

One skilled in the art readily appreciates that the present invention is well adapted to carry out the objectives and obtain the ends and advantages mentioned as well as those inherent therein. Particles, compositions, treatments, methods, kits, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

We claim:

1. A shaped particle for use in treating a bone deficiency wherein said particle is shaped for use in an array of particles interlocked with one another, comprising:
a center portion; and
at least four tapered extremities projecting from said center portion wherein said projections provide for interstitial spaces between adjacent extremities, each extremity having a base attached at said center portion, an opposite point, a length, and a circular transverse cross-sectional configuration, wherein said interstitial spaces of one said particle will accept at least one extremity of an adjacent said particle to facilitate interlocking of adjacent particles in said array,
wherein the particle is comprised of bone material.

2. The particle of claim 1 wherein at least three of said extremities lie in a plane.

3. The particle of claim 1 wherein said particle has six extremities.

4. A shaped particle for use in treating a bone deficiency wherein said particle is shaped for use in an array of particles interlocked with one another, comprising:
a center portion,
at least two noncurved extremities, and
at least three curved extremities projecting from said center portion wherein said projections provide for interstitial spaces between adjacent extremities, each extremity having a base attached at said center portion, an opposite point, a length, and a transverse cross-sectional configuration, wherein said interstitial spaces of one said particle will accept at least one extremity of an adjacent said particle to facilitate interlocking of adjacent particles in said array,
wherein the particle is comprised of a bone material.

5. A shaped particle for use in treating a bone deficiency wherein said particle is shaped for use in an array of particles interlocked with one another, comprising a multi-ring structure having at least four curved projections wherein said projections provide for interstitial spaces between adjacent said projections, and wherein said projections facilitate interlocking of adjacent particles in said array,
wherein the particle is comprised of a bone material.

6. The particle of claim 1 wherein said bone material is allograft bone material.

7. The particle of claim 6, wherein said allograft bone material is cortical-cancellous bone, cortical bone, cancellous bone, demineralized bone material, or mixtures thereof.

8. The particle of claim 7, wherein the demineralized bone material is fully demineralized, partially demineralized, or a mixture thereof.

9. The particle of claim 7, wherein the demineralized bone material is a powder.

10. The particle of claim 1 wherein said particle has maximum dimensions of about 3-10 millimeters.

11. The particle of claim 1 wherein said particle has a maximum dimensions of about 4-8 millimeters.

12. The particle of claim 1 wherein said particle has a maximum dimensions of about 4-6 millimeters.

13. The particle of claim 1 wherein said particle further comprises a biological agent.

14. The biological agent of claim 13 wherein said agent is selected from the group consisting of a growth factor, an antibiotic, a strontium salt, a fluoride salt, a magnesium salt, a sodium salt, a bone morphogenetic factor, an angiogenic factor, a chemotherapeutic agent, a pain killer, a bisphosphonate, a growth factor binding/accessory protein, a cell, and a bone growth agent.

15. The growth factor of claim 14 wherein said growth factor is selected from the group consisting of platelet derived growth factor (PDGF), transforming growth factor β (TGF-β), insulin-related growth factor-I (IGF-I), insulin-related growth factor-II (IGF-II), fibroblast growth factor (FGF), beta-2- microglobulin (BDGF II), nerve growth factor (NGF), epidermal growth factor (EGF), keratinocyte growth factor (KGF), and bone morphogenetic protein (BMP).

16. The antibiotic of claim 14 wherein said antibiotic is selected from the group consisting of tetracycline hydrochloride, vancomycin, cephalosporins, quinolone, and aminoglycocides.

17. The antibiotic of claim 16, wherein said quinolone is ciprofloxacin.

18. The antibiotic of claim 16, wherein said aminoglycocide is tobramycin or gentamicin.

19. The bone morphogenetic factor of claim 14 wherein said factor is selected from the group consisting of proteins of demineralized bone, demineralized bone matrix (DBM), bone protein (BP), bone morphogenetic protein (BMP), osteonectin, osteocalcin and osteogenin.

20. The angiogenic factor of claim 14, wherein said factor is monobutyrin, erucimide, synthetic thymosin Beta 4(TB4), synthetic peptide analogs to heparin binding proteins, nicotine, nicotinamide, spermine, angiogenic lipids, thrombin, a related analog/peptide of thrombin, dibutyrin, tributyrin, VEGF, butyric acid, or ascorbic acid.

21. The angiogenic factor of claim 14, wherein said factor is monobutyrin, erucimide, synthetic thymosin Beta 4(TB4), synthetic peptide analogs to heparin binding proteins, nicotine, nicotinamide, spermine, angiogenic lipids, thrombin, a related analog/peptide of thrombin, dibutyrin, tributyrin, VEGF, butyric acid, ascorbic acid, or derivatives thereof.

22. The growth factor binding/accessory protein of claim 14 wherein said factor is selected from the group consisting of follistatin, osteonectin, sog, chordin, dan, cyr61, thrombospondin, type IIa collagen, endoglin, cp12, nell, crim, acid-1 glycoprotein, and alpha-2HS glycoprotein.

23. The cell of claim 14 wherein said cell is selected from the group consisting of osteoblasts, endothelial cells, fibroblasts, adipocytes, myoblasts, mesenchymal stem cells, chondrocytes, multipotent stem cells, pluripotent stem cells and totipotent stem cells, and musculoskeletal progenitor cells.

24. The chemotherapeutic agent of claim 14 wherein said agent is selected from the group consisting of cis-platinum, ifosfamide, methotrexate and doxorubicin hydrochloride.

25. The pain killer of claim 14 wherein said pain killer is selected from the group consisting of lidocaine hydrochloride, bipivacaine hydrochloride, and non-steroidal anti-inflammatory drugs.

26. The pain killer of claim 25, wherein said non-steroidal anti-inflammatory drug is ketorolac tromethamine.

27. The array of claim 1 wherein said array contains multiple particles.

28. The array of claim 27 wherein said multiple particles are in a mixture of particles comprised of different materials.

29. The particles of claim 28 wherein said different materials are selected from the group consisting of bone material, ceramic, calcium salt, bioactive glass, polymer, polymer/ceramic composite, polymer/glass composite, and mixtures thereof.

30. The particles of claim 29, wherein the bone material is an allograft material.

31. The particles of claim 30, wherein the allograft material is demineralized bone material, cortical-cancellous bone, cortical bone, cancellous bone, or mixtures thereof.

32. The particles of claim 31, wherein the demineralized bone material is fully demineralized, partially demineralized, or mixtures thereof.

33. The particle of claim 1 wherein said treatment of a bone deficiency is selected from the group consisting of augmentation of bone, repair of bone, replacement of bone, improvement of bone, strengthening of bone and healing of bone.

34. The bone deficiency of claim 33 wherein said bone deficiency is selected from the group consisting of a fracture, break, loss of bone, weak bone, brittle bone, hole in bone, void in bone, disease of bone and degeneration of bone.

35. The disease of claim 34 wherein said disease is selected from the group consisting of osteoporosis, Paget's disease, fibrous dysplasia, osteodystrophia, periodontal disease, osteopenia, osteopetrosis, primary hyperparathyroidism, hypophosphatasia, fibrous dysplasia, osteogenesis imperfecta, myeloma bone disease and bone malignancy.

36. The array of claim 1 wherein said interlocking of said adjacent particles in said array provides adequate porosity to allow ingrowth from a host bone.

37. The array of claim 36 wherein said porosity is between about 40% and about 80%.

38. The array of claim 36 wherein said porosity is between about 50% and about 80%.

39. An array of shaped particles wherein said array comprises a plurality of shaped particles, said shaped particles comprising:
a center portion; and
at least four tapered extremities projecting from said center portion wherein said projections provide for interstitial spaces between adjacent extremities, each extremity having a base attached at said center portion, an opposite point, a length, and a circular transverse cross-sectional configuration, wherein said interstitial spaces of one said particle will accept at least one extremity of an adjacent said particle to facilitate interlocking of adjacent particles in said array of shaped particles, wherein said array of shaped particles provides for treating a bone deficiency,
wherein at least one of the particles is comprised of bone material.

40. An array of shaped particles wherein said array comprises a plurality of shaped particles comprising one or more shaped particles from the group consisting of:
a first shaped particle comprising a center portion and at least four tapered extremities projecting from said center portion wherein said projections provide for interstitial spaces between adjacent extremities, each extremity having a base attached at said center portion, an opposite point, a length, and a circular transverse cross-sectional configuration, wherein said interstitial spaces of one said particle will accept at least one extremity of an adjacent said particle to facilitate interlocking of adjacent particles in said array of shaped particles;
a second shaped particle comprising a center portion, at least two noncurved extremities, and at least three curved extremities projecting from said center portion wherein said projections provide for interstitial spaces between adjacent extremities, each extremity having a base attached at said center portion, an opposite point, a length, and a transverse cross-sectional configuration, wherein said interstitial spaces of one said particle will accept at least one extremity of an adjacent said particle to facilitate interlocking of adjacent particles in said array; and
a third shaped particle comprising a multi-ring structure having at least four curved projections wherein said projections provide for interstitial spaces between adjacent said projections, and wherein said projections facilitate interlocking of adjacent particles in said array,
wherein at least one of the particles is comprised of bone material.

41. A shaped particle for use in treating a bone deficiency wherein said particle is shaped for use in an array of particles interlocked with one another, comprising a multi-ring structure having at least four curved projections wherein said projections provide for interstitial spaces between adjacent said projections, and wherein said projections facilitate interlocking of adjacent particles in said array, wherein the particle is comprised of bone material.

42. The shaped particle of claim 41 wherein the angles between said curved projections are equal.

43. A composition for use in treating a bone deficiency comprising:
a suspension material; and
a shaped particle selected from the group consisting of
a first shaped particle comprising a center portion and at least four tapered extremities projecting from said center portion wherein said projections provide for interstitial spaces between adjacent extremities, each extremity having a base attached at said center portion, an opposite point, a length, and a circular transverse cross-sectional configuration, wherein said interstitial spaces of one said particle will accept at least one extremity of an adjacent said particle to facilitate interlocking of adjacent particles in said array of shaped particles;
a second shaped particle comprising a center portion, at least two noncurved extremities, and at least three curved extremities projecting from said center portion wherein said projections provide for interstitial spaces between adjacent extremities, each extremity having a base attached at said center portion, an opposite point, a length, and a transverse cross-sectional configuration, wherein said interstitial spaces of one said particle will accept at least one extremity of an adjacent said particle to facilitate interlocking of adjacent particles in said array;
a third shaped particle comprising a multi-ring structure having at least four curved projections wherein said projections provide for interstitial spaces between adjacent said projections, and wherein said projections facilitate interlocking of adjacent particles in said array, and
mixtures thereof, wherein the particle is comprised of bone material.

44. The suspension material of claim 43 wherein said suspension material is selected from the group consisting of starch, sugar, glycerin, blood, bone marrow, autograft material, allograft material, fibrin clot and fibrin matrix.

45. The suspension material of claim 43 wherein said suspension material is a binder capable of forming a gel.

46. The binder of claim 45 wherein said binder is selected from the group consisting of collagen derivative, cellulose derivative, methylcellulose, hydroxypropylcellulose, hydroxypropylmethyl cellulose, carboxymethylcellulose, fibrin clot, fibrin matrix, hyaluronic acid, chitosan gel, and a biological adhesive such as cryoprecipitate.

47. The suspension material of claim 43 wherein said material further comprises a biological agent.

48. The biological agent of claim 45 wherein said agent is selected from the group consisting of a growth factor, an antibiotic, a strontium salt, a fluoride salt, a magnesium salt, a sodium salt, a bone morphogenetic factor, an angiogenic factor, a chemotherapeutic agent, a pain killer, a bisphosphonate, growth factor binding/accessory protein, a cell, and a bone growth agent.

49. The growth factor of claim 48, wherein said growth factor is selected from the group consisting of platelet derived growth factor (PDGF), transforming growth factor β (TGF-β), insulin-related growth factor-I (IGF-I), insulin-related growth factor-II (IGF-II), fibroblast growth factor (FGF), beta-2- microglobulin (BDGF II), nerve growth factor (NGF), epidermal growth factor (EGF), keratinocyte growth factor (KGF), and bone morphogenetic protein (BMP).

50. The antibiotic of claim 48 wherein said antibiotic is selected from the group consisting of tetracycline hydrochloride, vancomycin, cephalosporins, quinolone, and aminoglycocides.

51. The antibiotic of claim 50, wherein said quinolone is ciprofloxacin.

52. The antibiotic of claim 50, wherein said aminoglycocide is tobramycin or gentamicin.

53. The bone morphogenetic factor of claim 48 wherein said factor is selected from the group consisting of proteins of demineralized bone, demineralized bone matrix (DBM), bone protein (BP), bone morphogenetic protein (BMP), osteonectin, osteocalcin and osteogenin.

54. The angiogenic factor of claim 48, wherein said factor is monobutyrin, erucimide, synthetic thymosin Beta 4(TB4), synthetic peptide analogs to heparin binding proteins, nicotine, nicotinamide, spermine, angiogenic lipids, thrombin, a related analog/peptide of thrombin, dibutyrin, tributyrin, VEGF, butyric acid, or ascorbic acid.

55. The angiogenic factor of claim 48, wherein said factor is monobutyrin, erucimide, synthetic thymosin Beta 4(TB4), synthetic peptide analogs to heparin binding proteins, nicotine, nicotinamide, spermine, angiogenic lipids, thrombin, a related analog/peptide of thrombin, dibutyrin, tributyrin, VEGF, butyric acid, ascorbic acid, or derivatives thereof.

56. The growth factor binding/accessory protein of claim 48 wherein said factor is selected from the group consisting of follistatin, osteonectin, sog, chordin, dan, cyr61, thrombospondin, type IIa collagen, endoglin, cp12, nell, crim, acid-1 glycoprotein, and alpha-2HS glycoprotein.

57. The cell of claim 48 wherein said cell is selected from the group consisting of osteoblasts, endothelial cells, fibroblasts, adipocytes, myoblasts, mesenchymal stem cells, chondrocytes, multipotent stem cells, pluripotent stem cells and totipotent stem cells, and musculoskeletal progenitor cells.

58. The chemotherapeutic agent of claim 48 wherein said agent is selected from the group consisting of cis-platinum, ifosfamide, methotrexate and doxorubicin hydrochloride.

59. The pain killer of claim 48 wherein said pain killer is selected from the group consisting of lidocaine hydrochloride, bipivacaine hydrochloride, and non-steroidal anti-inflammatory drugs such as ketorolac tromethamine.

60. The composition of claim 43 which further includes a clotting factor composition.

61. The clotting factor composition of claim 60 wherein said clotting factor composition comprises fibrinogen, thrombin, Factor XIII, or a combination thereof.

62. A method to treat a bone deficiency comprising the step of:
applying a shaped particle to a bone deficiency wherein said shaped particle is selected from the group consisting of
a first shaped particle comprising a center portion and at least four tapered extremities projecting from said center portion wherein said projections provide for interstitial spaces between adjacent extremities, each extremity having a base attached at said center portion, an opposite point, a length, and a circular transverse cross-sectional configuration, wherein said interstitial spaces of one said particle will accept at least one extremity of an adjacent said particle to facilitate interlocking of adjacent particles in said array of shaped particles;
a second shaped particle comprising a center portion, at least two noncurved extremities, and at least three curved extremities projecting from said center portion wherein said projections provide for interstitial spaces between adjacent extremities, each extremity having a base attached at said center portion, an opposite point, a length, and a transverse cross-sectional configuration, wherein said interstitial spaces of one said particle will accept at least one extremity of an adjacent said particle to facilitate interlocking of adjacent particles in said array; and
a third shaped particle comprising a multi-ring structure having at least four curved projections wherein said projections provide for interstitial spaces between adjacent said projections, and wherein said projections facilitate interlocking of adjacent particles in said array,
wherein the particle is comprised of bone material.

63. A method to treat a bone deficiency comprising the steps of:
combining a shaped particle with a suspension material wherein said particle is comprised of bone material and is selected from the group consisting of
a first shaped particle comprising a center portion and at least four tapered extremities projecting from said center portion wherein said projections provide for interstitial spaces between adjacent extremities, each extremity having a base attached at said center portion, an opposite point, a length, and a circular transverse cross-sectional configuration, wherein said interstitial spaces of one said particle will accept at least one extremity of an adjacent said particle to facilitate interlocking of adjacent particles in said array of shaped particles;
a second shaped particle comprising a center portion, at least two noncurved extremities, and at least three curved extremities projecting from said center portion wherein said projections provide for interstitial spaces between adjacent extremities, each extremity having a base attached at said center portion, an opposite point, a length, and a transverse cross-sectional configuration, wherein said interstitial spaces of one said particle will accept at least one extremity of an adjacent said particle to facilitate interlocking of adjacent particles in said array; and
a third shaped particle comprising a multi-ring structure having at least four curved projections wherein said projections provide for interstitial spaces between adjacent said projections, and wherein said projections facilitate interlocking of adjacent particles in said array; and
applying said combination to a bone deficiency.

64. A kit for the treatment of a bone deficiency comprising:
multiple shaped particles, wherein the particles are comprised of bone material and are selected from the group consisting of
a first shaped particle comprising a center portion and at least four tapered extremities projecting from said center portion wherein said projections provide for interstitial spaces between adjacent extremities, each extremity having a base attached at said center portion, an opposite point, a length, and a circular transverse cross-sectional configuration, wherein said interstitial spaces of one said particle will accept at least one extremity of an adjacent said particle to facilitate interlocking of adjacent particles in said array of shaped particles;
a second shaped particle comprising a center portion, at least two noncurved extremities, and at least three curved extremities projecting from said center portion wherein said projections provide for interstitial spaces between adjacent extremities, each extremity having a base attached at said center portion, an opposite point, a length, and a transverse cross-sectional configuration, wherein said interstitial spaces of one said particle will accept at least one extremity of an adjacent said particle to facilitate interlocking of adjacent particles in said array; and
a third shaped particle comprising a multi-ring structure having at least four curved projections wherein said projections provide for interstitial spaces between adjacent said projections, and wherein said projections facilitate interlocking of adjacent particles in said array.

65. The kit of claim 64, further comprising a suspension material.

66. The kit of claim 64 further comprising a biological agent.

67. The kit of claim 64 wherein the bone material is allograft material.

68. The kit of claim 64 further comprising a clotting factor composition.

69. The clotting factor composition of claim 68 wherein said clotting factor composition comprises fibrinogen, thrombin, Factor XIII, or a combination thereof.

70. A shaped particle for use in treating a bone deficiency wherein said particle is comprised of bone material and is shaped for use in an array of particles interlocked with one another, comprising:
a center portion;
at least two noncurved extremities; and
at least three curved extremities projecting from said center portion wherein said projections provide for interstitial spaces between adjacent extremities, each extremity having a base attached at said center portion, an opposite point, a length, and a transverse cross-sectional configuration, wherein said interstitial spaces of one said particle will accept at least one extremity of an adjacent said particle to facilitate interlocking of adjacent particles in said array.

71. The particle of claim 1, said particle manufactured by a method comprising the step of compressing a granulated bone material into said shape.

72. The method of claim 71, wherein said material further comprises a processing aid composition.

73. The method of claim 72, wherein said processing aid composition is selected from the group consisting of stearic acid, calcium stearate, magnesium stearate, natural polymer, synthetic polymer, sugar and combinations thereof.

74. The method of claim 72, wherein said processing aid composition is magnesium stearate or stearic acid.

75. The method of claim 73, wherein said natural polymer is starch, gelatin, or combinations thereof.

76. The method of claim 73, wherein said synthetic polymer is methylcellulose, sodium carboxymethylcellulose, or hydropropylmethylcellulose.

77. The method of claim 73, wherein said sugar is glucose or glycerol.

78. The method of claim 71, wherein said particle further comprises a biological agent.

79. The method of claim 78, wherein said biological agent is added to said material prior to said compaction step.

80. The method of claim 79, wherein said biological agent is added to said bone graft substitute subsequent to said compressing step.

81. The biological agent of claim 78, wherein said agent is selected from the group consisting of a growth factor, an antibiotic, a strontium salt, a fluoride salt, a magnesium salt, a sodium salt, a bone morphogenetic factor, an angiogenic factor, a chemotherapeutic agent, a pain killer, a bisphosphonate, a bone growth agent, an angiogenic factor, growth factor binding/accessory protein, a cell, and combinations thereof.

82. The method of claim 71, wherein the granulated bone material constituents are less than about 10 millimeters in diameter.

83. The method of claim 71, wherein the granulated bone material constituents are less than about 250 μm in diameter.

84. The method of claim 71, wherein the granulated bone material constituents are in a range of about 50 to 180 microns.

85. A method of manufacturing the particle of claim 1, comprising the steps of:
obtaining a bone material;
processing said material to produce a granulated bone material; and
subjecting said granulated bone material to a powder compaction process.

86. The method of claim 85, wherein said powder compaction process utilizes a withdrawal press, wherein said press comprises:
a stationary lower punch;
a moveable die;
a moveable upper punch; and
a moveable lower punch, wherein said stationary lower punch is contained within said moveable lower punch.

87. The method of claim 85, wherein said powder compaction process utilizes a withdrawal press, wherein said press comprises:
a stationary lower punch;
a moveable lower punch, wherein said stationary lower punch is contained within said moveable lower punch;
a stationary upper punch;
a moveable upper punch, wherein said stationary upper punch is contained within said moveable lower punch; and
a moveable die.

88. A method of manufacturing the particle of claim 1 from granulated bone material, said method comprising the steps of:
providing a stationary lower punch and a moveable lower punch which is vertically moveable about the stationary lower punch, a moveable die having at least one cavity and positionable generally above the stationary lower punch, and a moveable upper punch;
introducing the granulated bone material into the cavity;
positioning the moveable die generally above the stationary lower punch;
moving the moveable upper punch to pressably contact the material in opposition to the moveable lower punch and stationary lower punch; and
moving the moveable lower punch to pressably contact the material in opposition to the moveable upper punch,
whereby said moving steps form the material into the shaped bone graft substitute.

89. The method of claim 88, wherein the steps of moving the upper and lower punches effect a substantially uniform distribution of pressure within said material.

90. The method of claim 88, wherein at least one of the moving steps applies a force to the material in a range of about 0.2 to about 5 tons.

91. The method of claim 88, wherein at least one of the moving steps applies a force to the material in a range of about 0.2 to about 2 tons.

92. The method of claim 88, wherein at least one of the moving steps applies a force to the material in a range of about 0.5 to about 1 ton.

93. The method of claim 88, wherein said moving step of the moveable lower punch to the material is subsequent to the moving step of the moveable upper punch to the material.

94. A method of manufacturing a particle of claim 1 from granulated bone material, said method comprising the steps of:
introducing an amount of the granulated bone material into the cavity;
providing a lower punch assembly, an upper punch assembly, and a moveable die positionable generally above the lower punch assembly;
positioning the moveable die generally above the lower punch assembly;
moving the lower punch assembly in opposition to the moveable upper punch to pressably contact the material;
moving the upper punch assembly in opposition to the moveable lower punch to pressably contact the material,
whereby said moving steps form the material into the shaped bone graft substitute.

95. The method of claim 94, wherein the lower punch assembly is comprised of at least one of a stationary lower punch and a moveable lower punch vertically moveable about the stationary lower punch.

96. The method of claim 94, wherein the upper punch assembly is comprised of at least one of a stationary upper punch and a moveable upper punch vertically moveable about the stationary upper punch.

97. An apparatus for manufacturing a particle of claim 1 from granulated bone material, said apparatus comprising:
a stationary lower punch having a top surface;
a moveable lower punch vertically moveable about the stationary lower punch and having a top surface;
a moveable die having at least one cavity and positionable generally above the stationary lower punch; and
a moveable upper punch,
such that said moveable upper punch moves in opposition to said moveable lower punch to pressably contact the material contained within the cavity,
whereupon following pressably contacting the material by the moveable lower punch the top surface height of the lower moveable punch is above the top surface height of the stationary lower punch.

98. A method for manufacturing a bone graft substitute from granulated bone material, said method comprising the steps of:
providing:
a first punch assembly having a first contact surface configured to effect a relief profile onto a first surface of the granulated bone material;
a second punch assembly having a second contact surface; and
a moveable die having at least one cavity;
introducing the bone material into the cavity;
positioning the moveable die generally in alignment with the first punch assembly;
moving at least a portion of the first punch assembly to pressably contact the material in opposition to the second punch assembly to effect the desired relief profile on the first surface thereof; and
moving at least a portion of the second punch assembly to pressably contact the material in opposition to the first punch assembly,
whereby said moving steps form the material into the shaped bone graft substitute.

99. A method for manufacturing a particle of claim 1 from demineralized bone matrix material, said method comprising the steps of:
providing:
a first punch assembly having a first contact surface configured to effect a relief profile onto a first surface of the demineralized bone matrix material;
a second punch assembly having a second contact surface; and
a moveable die having at least one cavity;
introducing the demineralized bone matrix material into the cavity;
positioning the moveable die generally in alignment with the first punch assembly;
moving at least a portion of the first punch assembly to pressably contact the material in opposition to the second punch assembly to effect the desired relief profile on the first surface thereof; and
moving at least a portion of the second punch assembly to pressably contact the material in opposition to the first punch assembly,
whereby said moving steps form the material into the shaped bone graft substitute.

100. The method of claim 99, wherein the contact surface area of the first punch assembly is generally equivalent to a contact surface area of the second punch assembly such that the moving steps apply a substantially uniform pressure distribution to the material.

101. The method of claim 99, wherein the first punch assembly includes a stationary punch and a moveable punch, such that the steps of moving the first punch assembly includes moving the moveable punch to pressably contact the material.

102. The method of claim 99, wherein the second punch assembly includes a stationary punch and a moveable punch, such that the steps of moving the first punch assembly includes moving the moveable punch to pressably contact the material.

103. An apparatus for manufacturing a particle of claim 1 from a granulated bone material, said apparatus comprising:
a first punch assembly having a first contact surface having a profile configured to effect a relief profile onto a surface of the bone material;
a second punch assembly having a second contact surface, the second contact surface positioned in general alignment with the first contact surface; and
a moveable die having at least one cavity, the moveable die being positionable generally in between the first and second punch assemblies.
